SEQUENCE LISTING (19 sequences)

SEQ ID NO: 1 - DNA, 1626 bp, Rhodococcus equii
  CDS (1)..(1623): groEL gene
atggccaagatcatcgcgttcgacgaagaggcacgtcgcggcctcgagcgtggcctgaacagcctcgctgacgccgtcaaggtgacgctgggacccaagggtcgcaacgtcgtgctggagaagaagtggggcgcccccacgatcaccaacgacggtgtttccatcgccaaggagatcgagctcgaggacccctacgagaagatcggcgccgagctggtcaaggaggtcgccaagaagaccgacgacgtcgctggtgacggcaccacgacggctacggtcctggctcaggcgctcgtccgcgagggcctgcgcaacgtcgctgccggcgccaacccgctgggtctgaagcgcggcatcgagaaggccgtcgaggccgtcaccgccaagctgctcgacaccgccaaggaggtcgagaccaaggagcagatcgctgccaccgccgggatctcggcgggcgactccacgatcggcgagctcatcgccgaggcgatggacaaggtcggcaaggaaggcgtcatcaccgtcgaggagtcgaactccttcggcctgcagctcgagctcaccgagggtatgcgcttcgacaagggctacatctcgctgtacttcgcgaccgacgccgagcgtcaggaagcggtcctcgaggatccgtacatcctgctcgtcagctccaagatctcgacggtcaaggacctgctgccgctgctggagaaggtcatccaggccggcaagccgctgctgatcatcgccgaggacgtcgagggcgaggccctgtcgaccctggtcgtcaacaagatccgtggcaccttcaagtccgtcgccgtcaaggctccgggcttcggtgaccgccgcaaggcacagctcgccgacatcgccatcctcaccggtggcgaggtcgtcagcgaagaggtcggcctgtccctcgagaccgccggcatcgagctgctgggccgcgcccgcaaggtcgtcgtcaccaaggacgagaccaccatcgtcgagggtgccggcgacgccgacgccatcgccggtcgcgtcaaccagatccgcgccgagatcgaggcgtcggattcggactacgaccgtgagaagctgcaggagcgcctggccaagctggccggtggcgttgccgtcatcaaggccggcgctgccaccgaggtggagctcaaggagcgcaagcaccgcatcgaggacgccgtccgcaacgccaaggctgcggtcgaggagggcatcgtcgccggtggtggcgtggctctgctgcagtcctcgccggtcctggacgacctgaagctcgagggcgacgaggccaccggtgccaacatcgtcaaggttgctctcgaggcgccgctcaagcagatcgcgttcaacgcgggcctcgagcccggcgtcgtcgcggagaaggttcgcaacctgcccgccggcagcggcctcaacgccgccaccggtgagtacgaggacctgctggctgctggcatcaacgacccggtcaaggtcacccgctcggcgctgcagaatgctgcgtccatcgcggctctgttcctgaccaccgaggctgtcgtcgccgacaagccggagaaggccgctgctccgatgggcgacccgaccggtggcatgggcggcatggacttctga

SEQ ID NO: 2 - protein, 20 aa, Rhodococcus equii
  sequence containing antigenic determinant of VapA
TSLNLQKDEPNGRASDTAGQ

SEQ ID NO: 3 - protein, 11 aa, Rhodococcus equii
  sequence containing antigenic determinant of VapA
NLQKDEPNGRA

SEQ ID NO: 4 - DNA, 20 bp, Artificial Sequence
  Primer
caaggaggtcgagaccaagg

SEQ ID NO: 5 - DNA, 20 bp, Artificial Sequence
  Primer
gtgccgcggatcttgttgac

SEQ ID NO: 6 - protein, 6 aa, Chlamydia trachomatis
  Epitope for binding Hsp60 antibody
LTTEAL

SEQ ID NO: 7 - DNA, 25 bp, Artificial Sequence
  Primer
acggtaccatggccaagatcatcgc

SEQ ID NO: 8 - DNA, 24 bp, Artificial Sequence
  Primer
cgtcaagcttgaagtccatgccgc

SEQ ID NO: 9 - DNA, 29 bp, Artificial Sequence
  Primer
gaggatccatggagactcttcacaagacg

SEQ ID NO: 10 - DNA, 28 bp, Artificial Sequence
  Primer
gatgaattctaacaaccgaggctgagcg

SEQ ID NO: 11 - DNA, 24 bp, Artificial Sequence
  Primer
tcttcggatccgctaattaccggc

SEQ ID NO: 12 - DNA, 24 bp, Artificial Sequence
  Primer
ggaattcgcaccaatcctgttgcg

SEQ ID NO: 13 - DNA, 55 bp, Artificial Sequence
  Primer
aaccttcagaaagacgaaccgaacggtcgagcagagcgtcaggaagcggtcctcg

SEQ ID NO: 14 - DNA, 25 bp, Artificial Sequence
  Primer
ctatagaatagggccctctagacgg

SEQ ID NO: 15 - DNA, 56 bp, Artificial Sequence
  Primer
tgctcgaccgttcggttcgtctttctgaaggttggcgtcggtcgcgaagtacagcg

SEQ ID NO: 16 - DNA, 23 bp, Artificial Sequence
  Primer
gagacccaagcttggtaccatgg

SEQ ID NO: 17 - DNA, 29 bp, Artificial Sequence
  Primer
gaggatccatggagactcttcacaagacg

SEQ ID NO: 18 - DNA, 26 bp, Artificial Sequence
  Primer
gcctcgagggcgttgtgccagctacc

SEQ ID NO: 19 - DNA, 24 bp, Artificial Sequence
  Primer
cgtcaagcttgaagtccatgccgc

(19) United States
(12) Patent Application Publication
(10) Pub. No.: US 2005/0063984 A1
(43) Pub. Date: Mar. 24, 2005

(54) Antigenic peptide fragments of vapa protein, and uses thereof

(76) Inventors: Thiru Vanniasinkam, Adelaide (AU); Mary Barton, Adelaide (AU); Michael W Heuzenroeder, Adelaide (AU)

Correspondence Address: R. Neil Sudol/Henry D. Coleman;William J Sapone, Colelan Sudol Sapone, 708 Third Avenue 14th Floor, New York, NY, 10017-4104, US

(21) Appl. No.: 10/491,300

(22) PCT Filed: Oct. 25, 2002
(86) PCT No.: PCT/AU02/01460

(30) Foreign Application Priority Data
Oct. 26, 2001 (AU) PR8523

(51) Int. Cl. A61K 39/02; C07K 14/195

(52) U.S. Cl. 424/190.1; 530/350

(57) ABSTRACT

A chimeric GroEL protein is provided which includes a surface exposed to exogenous amino acid sequence, which comprises an antigenic determinant of, for example, a pathogenic micro-organism. The exogenous amino acid sequence might be inserted into a hydrophilic region of the GroEL protein to provide a means of exhibiting the antigenic determinant to elict an immune response specifically reactive to the antigenic determinant. This provides for a cellular bias to the elicited immune response and is thus likely to be particulary useful for intracellular parasites.

### FIELD OF THE INVENTION

This invention relates to a GroEL chimeric protein and a vaccine which can be used in a method of eliciting an immune response in a mammal to an antigen, in particular to enhancing an immune response to a microorganism or allergen by the providing a chimeric GroEL protein or nucleic acid with an inserted protein or nucleic acid of an immunogenic determinant for the microorganism or allergen.

### BACKGROUND OF THE INVENTION

Certain microbial diseases of mammals are appropriately prevented by vaccinating a population at risk. The form of the vaccine has traditionally been with killed, or attenuated live organism. This approach has not been successful for all pathogens because antigens presented at the time of infection may be masked or not present during mass preparation of the vaccines. Additionally there are risks associated with accidental exposure of the vaccinated population to live organism that were supposedly killed and adverse reactions to components of preparations made from whole organisms, in particular endotoxins.

To circumvent difficulties associated with the use of vaccines derived from whole microorganisms, a major push over several decades has been to identify specific antigenic determinants presented on the surface of infecting microorganisms, antibodies against which can reduce and preferably eliminate the risk of infection. The aim is to identify one or more major antigenic determinants which are stably present on the microorganism for interaction with antibodies for opsonisation and disposal by the immune system of the susceptible population; and to attempt to elicit an appropriate immune response to the one or more major antigenic determinant.

In this use of major antigenic determinants the hope is also to achieve a response that is appropriate for the microorganism concerned. Thus an immune response biased toward cellular immunity is preferred for a pathogen that is intracellular. Alternatively it might be referred to provide for mucosal immunity should that present a particular barrier to entry by the pathogen.

Several important proteins have been identified as major antigens, or indeed have been identified as causal, such as toxins and frimbrial proteins in enteric pathogens. Whole proteins, or modified proteins have been used as the basis of vaccination trials. In some cases however the immune response to immunisation programs have been rather low, and judged as inadequate to provide a protective effect.

An understanding of specific antigenic determinants have been further refined, and in particular in the case of some proteins to specific linear amino acid sequences. This is the case for microorganisms as well as for allergens. The hope is then to present these amino acid strings in a format that elicits an immune response in the susceptible population. These linear antigenic determinants are, in one approach, inserted into another protein so as to be exposed at the surface and accordingly result in a suitable immune response. There is uncertainty in the nature of the immune response elicited by immunisation by preparations containing such chimeric protein. One is not sure, for example whether the type of response will be appropriate or if a sufficiently strong immunity will be provided. Additionally an immune respone to just one antigenic determinant may not be sufficient to provide the protection required.

### SUMMARY OF THE INVENTION

It has been found that an enhanced immune response was elicited by presenting a chimeric protein into a target animal, the chimeric protein is the result of the insertion of an antigenic determinant of the virulence associated protein VapA into the GroEL2 protein of Rhodococcus equi.

This finding provides a promising approach to dealing with R. equi infections in foals, however it also provides a mechanism of enhancing an immune response for other infections by utilising chimeric GroEL proteins into which antigenic determinants, or haptenic antigens that are normally poorly antigenic are inserted. The data, in particular to DNA vaccination, show an enhanced Th1 response, which is indicative of a greater cellular immunity compared to humoural immunity, and thus the general approach may be particularly applicable to intracellular infections. This finding also has implications beyond microbial infections to eliciting immune responses more generally and can be applicable, for example, to eliciting a hypoimmune response to various antigens such as might be desired in the case of allergens.

Accordingly in a broad form of a first aspect the invention could be said to reside in a method of eliciting an immune response in a mammal against an antigenic determinant, the method including the step of providing to the mammal a chimeric protein by a route and in a form to elicit an immune reaction that reacts with the antigenic determinant, the chimeric protein being a GroEL protein, modification or analogue thereof having a surface exposed exogenous amino acid sequence inserted therein, said exogenous amino acid sequence reactive with antibodies specific to the antigenic determinant.

The invention is more particularly, however, applicable to microbial infection and accordingly in a broad form of a second aspect the invention could be said to reside in a method of eliciting an immune response in a mammal against an antigenic determinant of a microorganism, the method including the step of administering to the mammal a chimeric protein, the chimeric protein being a GroEL protein, modification or analogue thereof having a surface exposed exogenous amino acid sequence inserted therein, said exogenous amino acid sequence configured to elicit an immune response specifically reactive to the antigenic determinant of the microorganism.

In a broad form of a third aspect the invention could be said to reside in a chimeric protein, said chimeric protein being a GroEL protein, modification or analogue thereof having a surface exposed exogenous amino acid sequence inserted therein, the exogenous amino acid sequence configured to elicit an immune response specifically reactive to the antigenic determinant.

In a broad form of a fourth aspect the invention could be said to reside in a nucleic acid encoding a chimeric protein, said chimeric protein being a GroEL protein, modification or analogue thereof having a surface exposed exongenous amino acid sequence inserted therein, the exogenous amino acid sequence configured to elicit an immune response specifically reactive to the antigenic determinant.

In a broad form of a fifth aspect the invention could be said to reside in a compostion for eliciting an immune response in a mammal directed against a microorganism or allergen, the composition including a chimeric protein in a pharmaceutically acceptable carrier, the chimeric protein being a GroEL protein, modification or analogue therof having a surface exposed exogenous amino acid sequence inserted therein, said exogenous amino acid sequence reactive with antibodies specific to an antigenic determinant of the microorganism or allergen.

### BRIEF DESCRIPTION OF THE DRAWINGS

FIG. 1 Nucleotide and amino acid sequence [SEQ ID No 1] encoding GroEL2 of Rhodococcus equi.

FIG. 2. Physical map of pIMVS-Re2. The R. equi groEL2 gene was inserted into pET-28a (+) vector which expressed GroEL2 with 6 histidine residues at the C-terminus. The vector contains a kanamycin cassette (Kan^{r}) for the selection of transformants, T7 promoter and Lac operator (lacI) for induction of protein expression.

FIG. 3. Physical map of construct pcDNA3-Re1 (pcDNA3 containing R. equi groEL2 with modified Kozak sequence). Restriction sites used for cloning groEL2 are indicated. The vector contains an ampicillin resistance cassette (Amp^{r}) for antibiotic selection, the human cytomegalovirus immediate early promoter (Pcmv) and SV40 origin for episomal replication.

FIG. 4 Physical map of construct pcDNA3-Re2 (Vector pcDNA3 with Kozak sequence containing R. equi vapA). Restriction sites used for cloning vapA are indicated. The vector contains an ampicillin resistance cassette (Amp^{r}) for antibiotic selection, the human cytomegalovirus immediate early promoter (Pcmv) and SV40 origin for episomal replication.

FIG. 5 Physical map of pIMVS-Re3. The R. equi vapA gene was inserted into pET-28a (+) vector which expressed VapA with 6 histidine residues at the C-terminus. The vector contained a kanamycin cassette (Kan^{r}) for the selection of transformants, T7 promoter and Lac operator (lacl) for induction of protein expression.

FIG. 6 Schematic representation of overlap extension PCR performed to create the chimeric groEL2/vapA DNA vaccine construct pcDNA3-Re3. The construct pcDNA3-Re1 was used as the template for the first two PCR reactions, in which products containing the inserted VapA epitope were produced (broken lines indicate sequence of VapA epitope). The PCR products obtained from these reactions were then used as templates for the final reaction to produce the PCR product used to create vaccine construct pcDNA3-Re3.

FIG. 7 Amino acid sequence of R. equi GroEL2. Residues in bold indicate region of hydrophilic residues associated with an area within GroEL proteins significantly associated with immunogenicity (as described by other researchers) (Panchanathan, et al., 1998). The arrow indicates the point of insertion of the VapA immunogenic epitope NLQKDEPNGRA [SEQ ID No 3] into GroEL2.

FIG. 8 Physical map of construct pcDNA3-Re3 (Vector pcDNA3 with Kozak sequence containing groEL2/vapA epitope chimeric gene). Restriction sites used for cloning vapA are indicated. The vector contains an ampicillin resistance cassette (Amp^{r}) for antibiotic selection, the human cytomegalovirus immediate early promoter (Pcmv) and SV40 origin for episomal replication.

FIG. 9 Physical map of pIMVS-Re4. The chimeric groEL2/vapA gene was inserted into pET-28a (+) vector which expressed the protein with 6 histidine residues at the C-terminus. The vector contained a kanamycin cassette (Kan^{r}) for the selection of transformants, T7 promoter and Lac operator (lacI) for induction of protein expression.

FIG. 10 R. equi GroEL2 specific IgG1/IgG2a antibody response following immunization with pcDNA3 vector (control), pcDNA3-Re1, His-tagged GroEL2 protein, pcDNA3-Re1+pORF-mlLI2 or live R. equi vaccine. Antibody levels were determined 2, 4 and 6 weeks after initial immunization. Data are shown as mean and standard error. Data were analysed using the Wilcoxon (rank sum) two-sample test (P<0.05) (FIG. 10A) IgG1 response: all vaccine constructs elicited a statistically significant response compared to the control (FIG. 10B) IgG2a response: all vaccine constructs elicited a statistically significant response compared to the control. pcDNA3 vector (control) (−), pcDNA3-Re1 ♦, His-tagged GroEL2 protein ▴, pcDNA3-Re1+pORF-mIL12 ▪, live R. equi ATCC 33701 ★

FIG. 11 R. equi GroEL2 specific IgG2b antibody response and DTH response following immunization with pcDNA3 vector (control), pcDNA3-Re1, His-tagged GroEL2 protein, pcDNA3-Re1+pORF-mIL12 or live R. equi vaccine. IgG2b subclass antibody levels were determined 2, 4 and 6 weeks after initial immunization. DTH response was determined two weeks after the last boost. Data are shown as mean and standard error. Data were analysed using the Wilcoxon (rank sum) two sample test (P<0.05) (C) IgG2b response: all vaccine constructs elicited a statistically significant response compared to the control (D) Delayed type hypersensitivity (DTH) response: all vaccine constructs elicited a statistically significant response compared to the control

pcDNA3 vector (control) (−), pcDNA3-Re1 ♦, His-tagged GroEL2 protein ▴, pcDNA3-Re1+pORF-mIL12 ▪, live R. equi A TCC 33701 ★

FIG. 12 R. equi VapA specific IgG1/IgG2a antibody response following immunization with pcDNA3 vector (control), pcDNA3-Re2, His-tagged VapA protein, pcDNA3-Re2+pORF-mlLI2 or live R. equi vaccine. Antibody levels were determined 2, 4 and 6 weeks after initial immunization. Data are shown as mean and standard error. Data were analysed using the Wilcoxon (rank sum) two sample test (P<0.05) (FIG. 11A) IgG1 response: all vaccine constructs elicited a statistically significant response compared to the control (FIG. 11B) IgG2a response: all vaccine constructs elicited a statistically significant response compared to the control. pcDNA3 vector (control) (−), pcDNA3-Re2 ⋄, His-tagged Vap A protein ♦, pcDNA3-Re2+pORF-mIL12 ▪, live R. equi ATCC 33701 ★

FIG. 13R. equi VapA specific IgG2b antibody response and DTH response following immunization with pcDNA3 vector (control), pcDNA3-Re2, His-tagged VapA protein, pcDNA3-Re2+pORF-mlLI2 or live R. equi vaccine. IgG2b antibody levels were determined 2, 4 and 6 weeks after initial immunization. DTH response was determined two weeks after the last boost. Data are shown as mean and standard error. Data were analysed using the Wilcoxon (rank sum) two sample test (P<0.05) (FIG. 12A) IgG2b response: all vaccine constructs elicited a statistically significant response compared to the control (FIG. 12B) Delayed type hypersensitivity (DTH) response: all vaccine constructs elicited a statistically significant response compared to the control. pcDNA3 vector (control) (−), pcDNA3-Re2 ⋄, His-tagged VapA protein ♦, pcDNA3-Re2+pORF-mIL12 ▪, live R. equi ATCC 33701 ★

FIG. 14 R. equi GroEL2 specific IgG1/IgG2a antibody response following immunization with pcDNA3 vector (control), pcDNA3-Re3, His-tagged chimeric GroEL2/VapA protein, pcDNA3-Re3+pORF-mIL12 or live R. equi vaccine. Antibody levels were determined 2, 4 and 6 weeks after initial immunization. Data are shown as mean and standard error. Data were analysed using the Wilcoxon (rank sum) two sample test (P<0.05) (FIG. 14A) IgG1 response: all vaccine constructs elicited a statistically significant response compared to the control (FIG. 14B) IgG2a response: all vaccine constructs elicited a statistically significant response compared to the control. pcDNA3 vector (control) (−), pcDNA3-Re3 ⋄, His-tagged chimeric GroEL2/VapA protein ♦, pcDNA3-Re3+pORF-mlLI2 ▪, live R. equi ATCC 33701 ★

FIG. 15 R. equi GroEL2 specific IgG2b antibody response and DTH response following immunization with pcDNA3 vector (control), pcDNA3-Re3, His-tagged GroEL2/VapA protein, pcDNA3-Re3+pORF-mlLI2 or live R. equi vaccine. IgG2b subclass antibody levels were determined 2, 4 and 6 weeks after initial immunization. DTH response was determined two weeks after the last boost. Data are shown as mean and standard error. Data were analysed using the Wilcoxon (rank sum) two sample test (P<0.05) (FIG. 15A) IgG2b response: only the chimeric protein vaccine construct elicited a statistically significant response compared to the control (FIG. 15B) Delayed type hypersensitivity (DTH) response: all vaccine constructs elicited a statistically significant response compared to the control, pcDNA3 vector (control) (−), pcDNA3-Re3 ♦, His-tagged chimeric GroEL2/VapA protein ▴, pcDNA3-Re3+pORF-mIL12 ▪, live R. equi ATCC 33701 ★

### DETAILED DESCRIPTION OF THE INVENTION

By way of a shorthand notation the following three and one letter abbreviations for amino acid residues are used in the specification as defined in Table 1.

Where a specific amino acid residue is referred to by its position in the polypeptide of an protein, the amino acid abbreviation is used with the residue number given in superscript (i.e. Xaaⁿ)

TABLE 1

| | Three-letter | One letter |
|---|---|---|
| Amino Acid | Abbreviation | Abbreviation |
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

GroEL proteins were originally identified in E. coli as one of the host factors required for bacteriophage capsid assembly during a lytic infection. The groEL2 gene is highly conserved between species and has been used in phylogenetic research (Gupta, 1995, Gupta, 2000). Members of the GroEL family are found in all eubacterial cells as well as eukaryotic mitochondria and chloroplasts (Gupta, 1995).

GroEL is known to facilitate the correct folding of various bacterial proteins as well as prevent the aggregation of denatured proteins by an ATP dependent mechanism (Craig, et al., 1993). The GroEL protein is composed of 14 subunits, arranged in heptameric rings with a central cavity. This central cavity referred to as the ‘Anfinsen cage’ provides a shielded environment for the refolding of proteins (Ma, et al., 2000).

In most eubacteria, the groEL (L-large) gene that encodes a protein approximately 60-65 kDa in size is present in the groE operon together with a smaller protein (Hsp10) encoding groES (S-small) gene (Segal and Ron, 1996). Several organisms contain just one copy of the groEL gene in an operon (Segal and Ron, 1996). However, there are several organisms including Mycobacteria sp. (Rinke de Wit, et al., 1992) and notably α-proteobacteria, such as the nitrogen fixing soybean nodule bacterium, Bradyrhizobium japonicum, that possess two or more copies of GroEL encoding genes in their chromosome (Karlin and Brocchieri, 2000). Organisms such as Mycobacteria sp. and other actinomycetes contain two groEL genes. One of these is designated groEL2 and is usually not associated with a groES gene in a bicistronic operon. Similar to groEL1 which is associated with groES in an operon, groEL2 is also induced following heat shock and other physiological stress (Duchêne, et al., 1994, Mazodier, et al., 1991).

The expression of GroEL proteins are known to be upregulated during infection of a host in a range of bacterial pathogens (Noll, et al., 1999). Importantly, these proteins have been shown to be immunodominant antigens in both humoral and cell-mediated host responses against many bacteria, particularly with respect to intracellular pathogens such as Legionella pneumophila (Sampson, et al., 1986, Zügel and Kaufmann, 1999b).

GroEL2 appears to be transcribed at a much higher rate than groEL1 when the organism is subjected to environmental stress such as high temperature (de León, et al., 1997). In addition, the groEL2 encoded protein appears to be immunodominant over the groEL1 encoded protein, although antibodies to both proteins are observed during infections (Lathigra, et al., 1991. Rinke de Wit, et al., 1992, Shinnick, 1991).

GroEL has been used on its own as a means of immunising against Mycobacterium tuberculosis as a preventative for tuberculosis in humans (Lowrie et al., 1997, 1999)

GroEL has been used in conjugates to enhance the immune response in poorly immunogenic antigens Cohen et al U.S. Pat. No. 5,869,058.

The present invention utilises the immunogenic properties of GroEL by the insertion of an exogenous amino acid sequence that is reactive with antibodies to an antigenic determinant of a surface protein of the microorganism into one or more regions of the GroEL protein that lead to is surface exposure on the chimeric protein. The inventors are unaware of any other demonstration of an enhanced immune reaction using this approach.

This approach has a range of applications. In one specific aspect the application is limited to infection of foals by Rhodococcus equii, but in other broader aspects the invention is to a range of pathogens, other microorganism or indeed other antigenic determinants such as might be present in a range of allergens.

It is preferred that the GroEL into which the exogenous amino acid sequence carries at least a major antigenic determinant that is immunogenically identical to the GroEL carried by the species of micro-organism with respect of which an immune response is to be elicited. Not all of the major antigenic determinants of the species specific GroEL need be carried. The preference is therefore that the immune response elicited in the mammal will be directed in part also to GroEL antigenic determinants. The most efficient way of providing such a GroEL is to make the insertion of the exogenous amino acids into the GroEL of the microorganism for which immunity is desired, to form the chimeric protein. Thus for example where it is desired to induce an immune reaction in R. equi, then the GroE1 from R. equi is used in addition to an amino acid sequence derived from R. equi.

Whilst it is desirable that the specificity of the immune response is directed totally to major antigenic determinants, given the conservative nature of the GroEL it is likely that the species specificity of the GroEL will not particularly influence the manner in which the antigenic determinant within the exogenous amino acid sequence is presented in the mammal.

The GroEL forming the basis of the chimeric protein might be any one encoded by a pathogenic micro-organism. There microorganism might be selected from the group of micro-organisms comprising Listeria ivanovii, Listeria monocytogenes, Salmonella enterica, Bordatella species, Mycobacterium species, Nocardia Species, Shigella species, Enteropathogenic E. coli, Yersinia species, Legionella species, Francisella tularensis, Brucella species, Chlamydiae, Rickettsiae.

DNA sequences of many GroEL genes are known many are referred to in Gupta, 1995 and Gupta 2000, and in Richardon et al., (1998) and Saibil (2000). Specific examples of DNA/amino acid sequences are listed as follow Mycobacterium marinum (genbank U55831), Mycobacterium tuberculosis H37Rv (genbank AL021932), Mycobacterium bovis (genbank M17705), Mycobacterium avium (genbank AF281650), Tsukamurella tyrosinosolvens (genbank U90204) Rhodococcus equi (genbank AF233387), Streptomyces lividans (genbank X95971), Streptomyces albus (genbank M76658), Corynebacterium aquaticum (genbank AF 184092), Pseudomonas aeruginosa (genbank M63957), Helicobacter pylori (genbank X73840), Borrelia burgdorferi (genbank X65139).

Where organisms carry two groEL genes preferably the groEL gene is that gene expressing groEL2. Such organism might include Mycobacteria species and other actinomycetes and α-proteobacteria.

It is also anticipated that variations and modification of the GroEL proteins will also form a reasonable basis for chimeric proteins. It is known that conservative substitutions in proteins, in particular in inessential parts of the protein still permit function, and indeed the mammal into which these chimeric proteins are to be introduced do not depend on them for GroEL function, and therefore the substitutions at least in certain parts need not be structural. It is anticipated therefore that groEL genes that have had genetic modification such as point mutations, and rearrangements such as deletions, truncations, substitutions, inversion and duplication may still function to appropriately present the inserted exogenous amino acid sequence and preferably also one or more major GroEL antigenic determinant. For example the GroEL protein might include partial deletion of an existing hydrophilic region or amino acid string defining an antigenic determinant to so that the exogenous amino acid sequence can be partially or fully substituted therein. In this respect it is preferably that the chimeric protein is still able to form the double heptameric ring, to thereby provide for a substantial exposure of the exogenous amino acid sequence.

The exogenous amino acid sequence as indicated above is to be inserted in the GroEL amino acid sequence so as to be exposed to the surface of the chimeric protein. Thus the exogenous amino acid sequence is presented so as to be accessible to receptors responsible for inducing the desired immune response. More preferably as indicated above the exogenous amino acid sequence is exposed to the surface of the double heptameric ring structure into which GroEL is formed.

One approach to determining appropriate sites for insertion is to calculate from the predicted amino acid sequence of the protein concerned a hydrophobicity plot, and to insert the exogenous amino acid sequence into one or more of the hydrophilic regions. Thus it might be desired to select more than one site of insertion. Indeed where the microorganism concerned has more than one major antigenic determinant, it might thus be desired to form a multivalent insertion, to express two or more exogenous amino acid sequences providing two or more further antigenic determinants.

Another approach to this is to insert the exogenous amino acid sequences into the GroEL sequence known itself to be an antigenic determinant. These may be as identified in Panchanathan et al., (1998). This latter approach however may not necessarily be preferred because it may be more preferable to have both the major existing antigenic GroEL determinants as well as that provided by the exogenous amino acid sequence so that two antigenic determinants are presented for an immune response.

With respect of the Rhodococcus equi GroEL, the hydrophilic regions might be selected from one or more of the following: —V26-S54, V73-T90, G109-A144, M191-L246, R270-1290, G342-A397 and V415-N468

More particularly this might be M191-L246.

These or similar hydrophilic regions in other GroEL protein can be predicted using the methods of Hopp and Woods. (1981) which method can also be used to predict which amino acids might be an antigenic determinant.

Assistance in that regard can also be had to the extensive work that has been made in amino acid/nucleic acid comparison and model work for GroEL (Richardon et al., 1998; Saibil, 2000). Using these models and/or protein sequence comparisons one can make a relatively informed calculation as to one or more sites most likely to provide for good presentation of the exogenous amino acid sequence. Additionally the above two references can assist in selecting an appropriate GroEL sequence to use for with a particular micro-organism or other target antigen.

The insertion might be in the form of a direct insertion into the existing sequence. Thus should the exogenous amino acid sequence be 11 amino acids long the chimeric protein will be 11 amino acids longer than the GroEL on which the chimeric protein is based. Alternatively deletion of GroEL of some amino acids might be effected in addition to the insertion of the exogenous amino acid sequence, to keep the size of any surface exposed loop down or perhaps the same size as they originally were.

Selection of the exogenous amino acids is anticipated to be quite important. Typically it is anticipated that they would represent linear antigenic determinants. Antigenic determinants on the surface of a protein are those features that are capable of binding an antibody. At times there is sufficient binding to a sequence of amino acids in a linear string, such that the string of amino acids presented will elicit binding by an antibody. These antigenic determinants are known as linear antigenic determinants. Alternatively more than one string of consecutive amino acid sequences are required to bind an antibody, these more complex antigenic determinants arise where antibodies recognised amino acids adjacent to one another on the surface of a protein but by reason of the folding of the primary amino acid sequence are not adjacent on the same linear sequence. The present invention is most particularly concerned with the presentation of the more simple linear antigenic determinants. Although where perhaps two strings of amino acids sequences form an antigenic determinants and the spacing of the adjacent loops in the GroEL matches that of the original antigenic determinant the present invention may be adapted for that purpose.

The length of linear antigenic determinant varies considerably and may range from three or four amino acid to about 25 amino acids.

The amino acid sequence might be selected to be reactive with a major antigenic determinant of a pathogenic micro-organism. It is found that with infections of a particular strain of micro-organisms that commonly an immune response is directed to just a few antigenic determinants. One antigenic determinant may in fact dominate. Additionally there might some minor antigenic determinant that are recognised. Generally the major antigenic determinant are those that are more accessible to cells of the immune system including those responsible for recognising and disposing of infectious micro-organisms. It is desirable to induce immunity as against major antigenic determinant because it is anticipated that these will provide, in a vaccine, for a better protective effect, or in the case of an allergen provide for more effective tolerance. Indeed in the case of inducing tolerance to an allergen it is anticipated that the antigenic determinant used will be the one to which the individual concerned has an allergic reaction.

The exogenous amino acids sequence might therefore be selected from a large range of currently identified major antigenic determinants. They might be selected for example from the following micro-organisms. Listeria ivanovii, Listeria monocytogenes, Salmonella enterica, Bordatella species, Mycobacterium species, Nocardia Species, Shigella species, Enteropathogenic E. coli, Yersinia species, Legionella species, Francisella tularensis, Brucella species, Chlamydiae, Rickettsiae.

Other examples of suitable exogenous amino acids sequences contemplated by the present invention are as follows. They may be derived from viruses such as rhinoviruses, rotavirus, retroviruses, polivirus. Suitable antigenic determinants for HIV might be those identified by Enshell-Seijffers et al., (FASEB 2001 15; 2012-2020). Hepatitis C virus such as identified for the E2 glycoprotein by Bugli et al., (J. Virol. 2001 75:9986-9990). Hepatitis delta virus identified by Fiedler and Roggendorf (Intervirology (2001) 44:154-161).

Similarly these might be for vaccination against various allergens for example certain pollen antigens identified by Focke et al., (FASEB (2001) 15:2042-2044).

Other amino acid sequences suitable for this invention might include those referred to in the review of random peptide libraries by Irving, Pan and Scott (Current Opinions in Chem Biol (2001) 5:314-324), or in the review by Partido (2000, Current Opinions in Molecular Therapy 2:74-79).

Indeed the exogenous amino acid sequence might additionally be desired to be a GroEL sequence. Thus, for example it might be desired to provided for an enhanced immune reaction by providing for more than one copy of a GroEL specific antigenic determinant.

Rhodococcus equi is an encapsulated and rod shaped, Gram positive bacterium that is considered to be a soil saprophyte that survives well in the soil environment. R. equi has long been considered a pathogen in horses principally in foals fewer than 6 months old (particularly 1-3 months old). Infection by the organism is accompanied by extra-pulmonary manifestations, causes a pyogranulomatous pneumonia, often such as bacteraemia, lymphadenitis, meningitis and enteritis (Barton and Hughes, 1980; Giguere and Prescott, 1997; Takai, 1997). Infections are often fatal if untreated. Apart from causing disease in horses, R. equi also causes infections in cattle, pigs and goats (Barton, 1992). R. equi is also known to cause severe pulmonary and disseminated disease in immuno-compromised humans, particularly AIDS patients (Capdevila et al., 1997).

In Australia most equine R. equi infections occur in summer (December to February) when the age of the foals as well as the warm and dry environmental conditions make the animals more susceptible to infection (Barton and Hughes, 1984).

Many vaccine candidates have been tested for the prevention of R. equi infection in foals. Vaccine candidates have predominantly been protein subunit or whole cell preparations. A vaccine preparation known as ‘Rhodovac’ developed by Clínica Equina (Capitán Sarmiento, Argentina), contains high concentrations of soluble virulent R. equi antigens including VapA (Becú, et al., 1997). Other VapA containing antigen preparations have also been developed (Prescott, et al., 1997a). In addition, a range of vaccine preparations comprising killed or live R. equi (Prescott, et al., 1997b, Varga, et al., 1997) have also been tested.

R. equi produces a range of putative virulence factors such as cholesterol oxidase, phospholipase C and Iccithinase (Smola et al. 1994). However one of the more important putative virulence factors is considered to be a 17 kDa virulence associated protein (VapA) which is plasmid encoded. This protein is known to be produced by up to 90% of equine clinical isolates of R. equi. Although VapA producing strains are widespread among disease causing isolates, recent work has shown that VapA protein alone is not sufficient to cause disease in foals and that other as yet unknown plasmid borne factors are likely to be involved (Giguere et al., 1999). The role of VapA in virulence is yet to be elucidated, although there is strong evidence to suggest that the plasmid encoding the protein may play an important part in the survival of the organism within macrophages (Hondalus and Mosser, 1994).

In one specific form the exogenous amino acids might be the antigenic determinant found to be dominant with respect of Rhodococcus equi being a part of the VapA protein (Vanniasinkam et al., 2001).

A putative 20 amino acid region of the VapA protein that is recognised by antibodies in the sera of horses infected with R. equi has been identified as TSLNLQKDEPNGRASDTAGQ [SEQ ID No 2], although it will be understood that the minimal region for antigenic recognition may be further defined within the identified sequence, or additionally the identified sequence may contain two or more separate adjacent epitopes. Thus the amino acid sequence may be any peptide that is capable of mimicking this region in so far as providing VapA specific immunogenicity. Therefore the peptide may be part of a larger peptide that contains the amino acid sequence TSLNLQKDEPNGRASDTAGQ [SEQ ID No 2] of the present invention, as well as one or more of the amino acids either side of that sequence in the native VapA protein.

Therefore in one form of the this aspect the amino acid sequence has 5 or more amino acid residues and contains all or part of the sequence TSLNLQKDEPNGRASDTAGQ [SEQ ID No 2], or immunologically active derivative or analogue thereof. Preferably the peptide contains 7 to 30 amino acid residues, and more preferably 10 to 12 amino acid residues. Most preferably the peptide contains the sequence NLQKDEPNGRA [SEQ ID No 3].

Whether a peptide of the present invention provides for VapA specific immunogenicity can be determined routinely by following the procedures set out in (Vanniasinkam et al 2001).

The peptide in this aspect of the invention may also be homologous to any of the abovementioned peptides provided that the peptide provides for VapA specific immunogenicity. In this context, a peptide is considered homologous to a peptide of the present invention when it is immuno cross-reactive with antibodies specific for the R. equi VapA protein. It will be recognised by those skilled in the art that some amino acid sequences within the peptide can be varied without significant effect on the structure or function of the peptide. Thus for instance it is anticipated that ‘type’ amino acid substitutions still retain immuno cross reactivity and as such a neutral amino acid may be conservatively substituted with another neutral natural or non-natural amino acid, an acidic amino acid may be conservatively substituted with a natural or non-natural acidic amino acid, a hydrophilic amino acid may be substituted with another hydrophilic amino acid, and so on, provided that the immunological function of the peptide is not altered by the substitution.

Typically seen as conservative substitutions are the replacement of one for another among the aliphatic amino acids Ala, Val, Leu and Ile; interchange of the hydroxyl residues Ser and Thr; exchange of the acidic residues Asp and Glu; substitutions between the amide residues Asn and Gin; exchange of the basic residues Lys and Arg; and replacements among the aromatic residues Phe and Tyr. Preferably the homologous peptide shares 50% homology with a peptide of the present invention, more preferably shares 70% homology, and most preferably shares 90% homology.

Generally the insertion will be achieved at the nucleic acid level. Using purified DNA of a vector encoding the GroEL protein, synthesizing a DNA sequence encoding the exogenous amino acid sequence, cutting the DNA encoding the GroEL protein at the site of insertion using a restriction endonuclease, ligating in the synthetic sequence, and isolating the recombinant DNA molecule so formed and introducing it into an appropriate host or vector to amplify the DNA for a DNA vaccine or for the production of a recombinant protein preparation.

It is found that the cellular immunity (Th1 immunity) is enhanced by the use of this invention. Accordingly it is anticipated that this may be a particularly useful approach to vaccinating for intracellular pathogens. To further-bias the immune reaction in favour of Th1 immunity it might be desired to provide the vaccine as a nucleic acid vaccine.

There are various strategies, which may improve the protective efficacy of the vaccines of this invention, and many of them are well known including the use of various adjuvants. Research has shown that the co-administration of immunostimulatory molecules, such as TL-18 (Kim, et al., 2001), together with a pathogen specific antigen encoding DNA vaccine may be used to enhance a Th1 type protective response. This approach may be useful to improve the level of Th1 type immunity observed with the vaccines used in this study, in order to induce a sufficient immune response that would confer protection against R. equi. Other strategies to improve the protective efficacy of the vaccine would include the development of multi-epitope vaccines that contain other R. equi genes in addition to groEL2 or the use of a prime-boost strategy to immunise the host (Ramshaw and Ramsay, 2000). Il-12 is also recognised for its role in maintaining long term cell-mediated immunity (Park and Scott, 2001). Therefore coadministration of IL-12, as a DNA vaccine or recombinant protein, in conjunction with a suitable primary vaccine may enhance protection against R. equi in the host.

The chimeric protein might be admininistered following purification of the protein and forming a vaccine composition that is administered to the mammal to elicit an immune reaction. The purification might be by known methods. The chimeric protein will be encoded by any one of a number of known expression vectors that is introduced into an expression microorganism. Purification by known methods follows fermentation. The purified or semi purified protein can then be administered.

The administration might be parenteral such as subcutaneously, intramuscularly, or it might simply presented to a mucosal surface, perhaps by pulmonary administration or alternatively administration might be intraperitonealy. The mucosal administration may be aimed at inducing local mucosal immunity to provide a barrier to entry by the organism concerned.

Preferably the chimeric protein is administered in pharmaceutical dosage form as a composition or formulation comprising an immunogenically effective amount of the chimeric protein. The amount of chimeric protein administered will vary depending on the pharmacokinetic parameters, severity of the disease treated or immunogenic response desired Doses may be set by a physician or veterinarian considering relevant factors including the age, weight and condition of the vertebrate including, in the case of immunogenic dosage forms, whether the vertebrate has been previously exposed to the microorganism responsible for the disease to be vaccinated against as well as the release characteristics of the peptide from pharmaceutical dosage forms of the present invention.

The composition may be injected or may be added to a pharmaceutically acceptable carrier as will be apparent to those skilled in the art and as set out in “Remington's Pharmaceutical Sciences”, Sixteenth Edition, Mack Publishing Co, 1980, and include water and other polar substances, including lower molecular weight alkanes, polyalkanols such as ethylene glycol, polyethylene glycol and propylene glycol as well as non-polar carriers.

The method of administering the vaccine may vary and could include intravenous, buccal, oral, transdermal and nasal as well as intramuscular or subcutaneous administration.

Preferably, the vaccine is administered by inhalation which may then set up local immunity. Alternatively the vaccine may be administered using other forms of mucosal priming.

In particular with the GroEL/vapA chimeric protein it might be desired to administer to the pulmonary system, perhaps as an aerosol, because that represents the transmission route of the pathogenic organism.

The vaccine might be provided in a composition using the chimeric protein, however, in another form the chimeric protein can be provided to the mammal by the injection of a nucleic acid encoding the chimeric protein preferably carried in a suitable nucleic acid vector. In that form the nucleic acid, usually DNA vector, is typically introduced intramuscularly, by published methods. Some of the nucleic acid is introduced intracellularly to transform a cell. The transformed cell then expresses the chimeric protein which is presented either on the cell surface to induce the immune reaction or is presented on senescence of the transformed cell to elicit an immune reaction.

DNA vaccines have been used for the induction of long-term cellular immunity for the prevention of bacterial and viral infections in large animals such as cattle (Babiuk, et al., 1998, Chaplin. et al., 1999). Lowrie et al., 1997. 1999 have used this approach to immunise against tuberculosis utilising, a GroEL based DNA vaccine. This might also require assistance with a substance such as Bupivacaine to assist with uptake of the DNA.

Alternatives for enhancing DNA vaccine delivery include methods of vaccine delivery such as gene gun inoculation (Yoshida, et al., 2000), the use of attenuated bacteria as the vaccine carrier (Dietrich, et al., 2001) or electrotransfer of the plasmid (Bachy, et al., 2001). In addition, the co-administration of adjuvants such as Th1 response-promoting cytokines or cationic mannan-coated liposomes (Toda, et al., 1997) could also be tested. Alternatively, the plasmid DNA could be administered as a supercoiled molecule (minicircle) devoid of origin of replication and antibiotic resistance cassettes. Minicircles are smaller and potentially safer than many currently used vaccine vectors and importantly have been shown to exhibit a high level of expression in vivo (Darquet, et al., 1999).

Potential DNA vaccines against various bacterial pathogens, particularly intracellular pathogens such as Mycobacterium tuberculosis (Lowrie, et al., 1997) and Chlamydia psittaci (Vanrompay, et al., 1999) have been developed. It is postulated that since intracellular pathogens in generally require a Th1 type response for protective immunity, a DNA vaccine approach, which may be used to elicit a Th1 response, may be potentially more useful than subunit vaccines (Strugnell, et al., 1997) or attenuated live vaccines which can sometimes be subject to variability in efficacy as has been observed with Mycobacterium bovis based vaccines (Behr and Small, 1997). Often, genes used in DNA vaccines are selected following the identification of immunodominant antigens and the genes encoding them. Some of the proteins encoded by these vaccine candidates genes have included heat shock proteins (Lowrie, et al., 1997) (Svanholm, et al., 2000), secreted antigens (Kamath, et al., 1999) and a range of other immunogens such as outer membrane proteins (Pal, et al., 1999).

The present invention is further illustrated by the following exemplification which is not intended to be limitng in any way.

### EXAMPLES

Methods

General molecular biological methods were employed to undertake the microbiological techniques and molecular miological techniques are as generally set out in Sambrook et al., 1989.

### Example 1

### Cloning and Sequencing of R. equi of Groel

Strains and Plasmids

R. equi ATCC 6939 was used for the cloning and sequencing of groEL2. The vector pET-28a(+) (Novagen) was used for the production of histidine (His)-tagged GroEL2 in E. coli BL21 (DE3).

Bacterial Growth Conditions

Bacteria were grown in L-broth for protein expression. Columbia agar was used for the propagation of transformants. E. coli DH5α was used as a host for recombinant plasmids for the cloning of groEL2 and E. coli BL21 (DE3) was used for the expression of His-tagged GroEL.

PCR amplification of a groEL2 gene containing fragment of the R. equi chromosome Oligonucleotide primers were designed to amplify a fragment of the R. equi groEL2 gene. Sequences of these primers were based upon regions of homology between the published sequences of the groEL2 of Mycobacterium tuberculosis H37 Rv (GenBank Accession No: AL021932) and groEL of Tsukamurella tyrosinosolvens (GenBank Accession No: U90204). (these genes were chosen based upon 16S r-RNA studies indicating that Tsukamurella and Mycobacterium sp. are closely related to R. equi (Ruimy et al., 1995) and therefore likely to possess a groEL2 gene highly similar to the R. equi groEL2 gene).

The forward primer used was 5′-CAAGGAGGTCGAGACCAAGG-3′ [SEQ ID No 4] and reverse primer was 5′-GTGCCGCGGATCTTGTTGAC-3′ [SEQ ID No 5]. PCR amplification was carried out, using an annealing temperature of 64° C. and chromosomal DNA from R. equi as the template. The PCR product was sequenced and Digoxigenin labelled (Boehringer Mannheim, Germany). The labelled product was then used to probeR. equi chromosomal DNA digested separately with the following restriction enzymes in Southern blot analysis: SacI, XbaI, SmaI, EcoRI, BamHI, NsiI, HindIII, KpnI and SphI. Fragments of differing lengths were identified in R. equi chromosomal DNA depending upon the digesting enzyme. A single SphI fragment 4.7 kb in size was identified and cloned into pGEM-7Zf(−) (construct was designated pIMVS-Re1) and the nucleotide sequence of the R. equi insert determined.

Sequencing of pIMVS-Re1 was performed using ABI Prism Big Dye chemistry (PE Applied Biosystems).

Expression of GroEL2 in R. equi

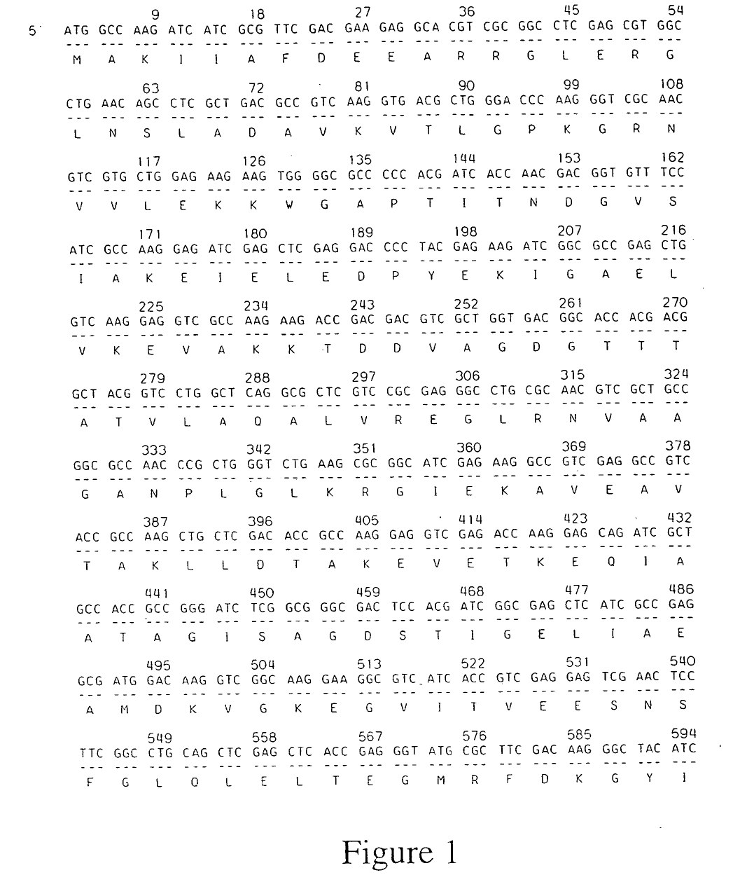

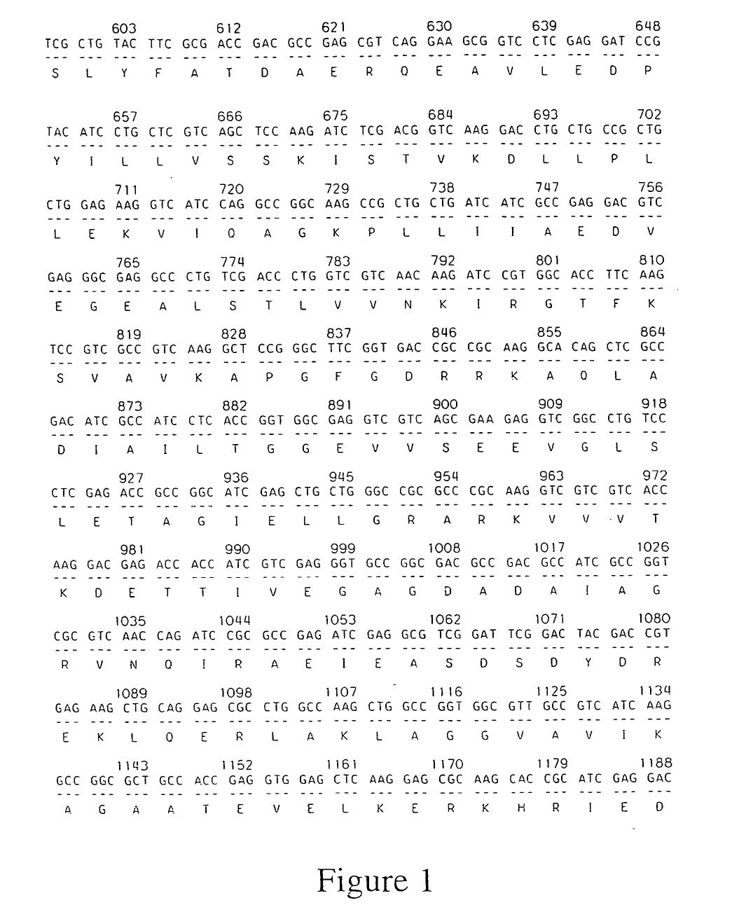

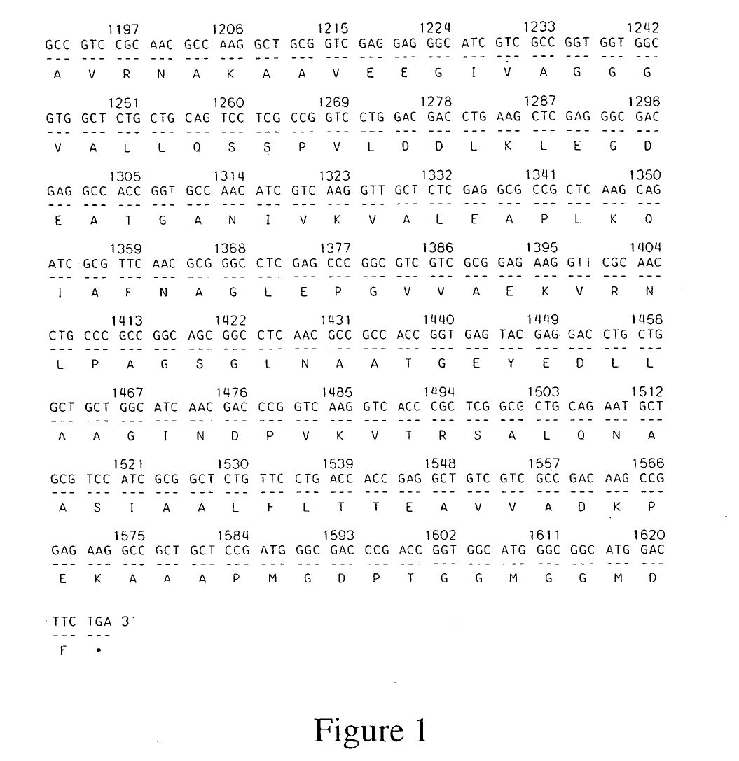

Two 10 ml aliquots of R. equi were grown overnight at 30° C. in L-broth with shaking. The following morning each aliquot culture was incubated at 30° C. or 42° C. for two hours with shaking. The culture was then centrifuged at 17, 000 g for 15 mins and pellet was resuspended in 1 ml 1×PBS and sonicated for 1 min and prepared for western immunoblot analysis using the Chlamydia trachomatis Hsp60 monoclonal antibody (Affinity Bioreagents Inc., CO, USA). This antibody is specific for amino acids 517 to 522 of the C. trachomatis HSP60 amino acid sequence and is known not to cross react with E. coli HSP60. The first five residues of the immunoreactive epitope (LTTEAL) [SEQ ID No 6] of the monoclonal antibody were identical to the amino acid residues 512 to 516 of the R. equi GroEL2 sequence (FIG. 1), and was therefore predicted to detect this protein when used in a western immunoblot analysis.

Sequence analysis was performed using GeneBase version 1.0 (Applied Maths, Kortrijk, Belgium) and BLASTX version 2.0 (Altschul, et al., 1997). The promoter region of the groEL2 gene was analysed using, the Berkeley Drosophila Genome Project promoter prediction website (www.fruitfly.org).

Subcloning of the R. equi groEL2 Gene

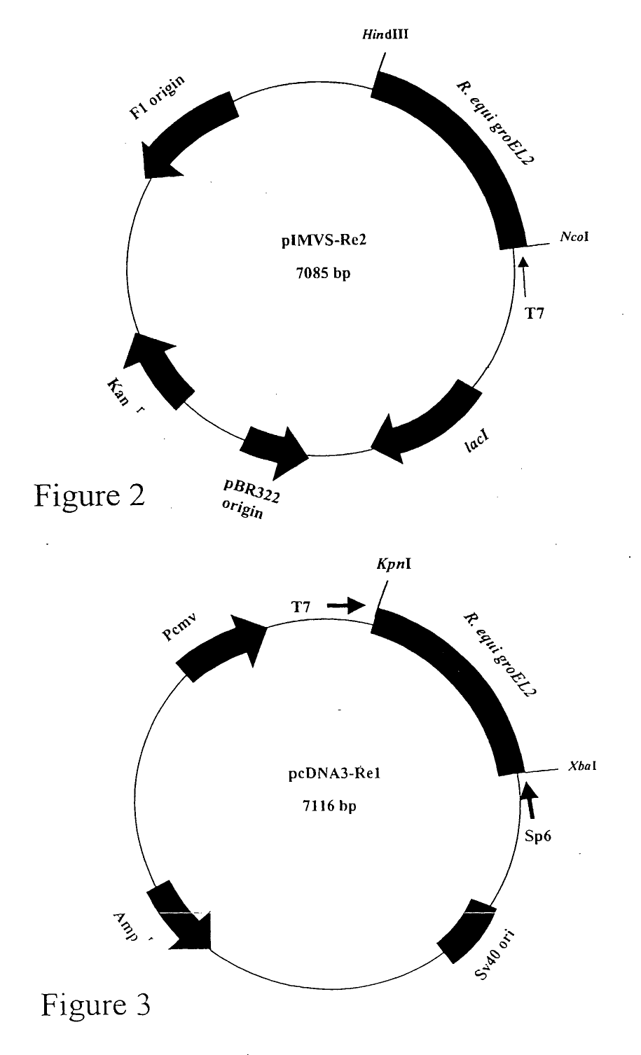

The groEL2 gene was PCR amplified using a forward primer 5′-ACGGTACCATGGCCAAGATCATCGC-3′[SEQ ID No 7] containing an introduced NcoI site (underlined) and reverse primer 5′-CGTC\overline{AAGCTT}GAAGTCCATGCCGC-3′[SEQ ID No 8] containing an introduced HindIII site (underlined) for cloning into pET-28a (+) (Novagen) expression vector. PCR was performed under standard conditions using DyNAzyme™ EXT DNA polymerase, an annealing temperature of 61° C. using a CsCl gradient purified preparation of pIMVS-Re1 as template. The PCR product and vector were separately digested with NcoI and HindIII and ligated to create construct pIMVS-Re2 (FIG. 2).

Production of a C-terminal His-Tagged GroEL2 Protein

Plasmid pIMVS-Re2 was transformed into E. coli BL21 (DE3) and the His-tagged GroEL2 protein was expressed and purified using Ni²⁺-NTA agarose (Qiagen) by the following method.

The clone containing pIMVS-Re2 was grown overnight in 4 ml L-broth containing 50 μg/ml kanamycin. This culture was added to 200 ml of L-broth containing 50 μg/ml kanamycin and was incubated with shaking for 3 hours. Protein production was induced with the addition of IPTG to the final concentration of 1 mM and the culture was grown for another 4 hours under the same conditions. The culture was then centrifuged at 3000 g and the pellet was stored at −20° C. overnight. The following day, the pellet was resuspended in 10 ml lysis buffer (20 mM Tris, pH8 and 1001 nM NaCl) and sonicated using 5, 15 second pulses. The solution was centrifuged at 17, 000 g for 15 mins. The supernatant was added to 1 ml Ni-NTA agarose that had been washed twice in lysis buffer (10 ml buffer added to the Ni-NTA agarose and centrifuged at 17, 000 g for 1 min). The solution was mixed on a rotary mixer (200 rpm) for 2 hours. The solution was then loaded on to a 5 ml column (Qiagen) and the column flow-through was removed. The Ni-NTA slurry deposited in the column was washed twice with 5 ml lysis buffer (buffer was added to the column and allowed to empty by gravity). A 500 μl aliquot of 250 mM imidazole solution was added to elute the protein from the NI-NTA slurry (performed twice), the protein eluate was stored in 100 μl aliquots at −20° C. until required.

Whole cell and protein preparations were separated on a 10% SDS PAGE.

N-terminal Sequence Analysis of C-Terminal 6× His-Tagged GroEL2

The 100 μl of purified His-tagged GroEL2 protein was separated on 10% SDS-PAGE and transferred to polyvinylidene difluoride membrane (Immobilon-P, Millipore, Mass., USA). The protein was then subjected to N-terminal amino acid sequencing by the Edman Degradation method (the sequencing was carried out by the Australian Proteome Analysis Facility, Macquarie University, NSW, Australia).

Results

The oligonucleotide primers amplified a 402 base pair (bp) PCR product from R. equi. This PCR product was found to be partially homologous to the groEL2 sequence of Mycobacterium avium and Mycobacterium paratuberculosis (P=6×10⁻⁶¹) and was used as a probe in Southern hybridisation to identify a transformant possessing a fragment of the R. equi genome containing the putative groEL2 gene.

A 4.713 kb fragment was found to contain a groEL2 gene (Genbank Accession No: AF233387) which was 1623 bp long and encoded a protein with a deduced molecular weight of 56543.5 Da. The gene had a high G+C content of 68% which is not surprising as R. equi is known to possess a GC rich genome (Goodfellow, 1987).

Western immunoblot analysis indicated that R. equi heat shocked at 42° C. produced a protein approximately 60 kDa in size, which was not detected in the culture grown at 30° C.

Homology of R. equi GroEL2 to Similiar Proteins in the Database

The inferred R. equi GroEL2 protein was found to be most closely related, with approximately 90% identity, to the GroEL2 proteins of Mycobacterium tuberculosis.

Mycobacterium leprae, Mycobacterium avium and Tsukamurella tyrosinosolvens. It was also related to the GroEL2-like proteins from other Gram positive actinomycetes such as Streptomyces albus, Streptomyces lividans and Streptomyces coelicor (Table 2). The R. equi GroEL2 was found to be less homologous (identity of approximately 60-69%) to GroEL1 sequences of other actinomycetes and was approximately 60% identical to GroEL sequences of organisms such as E. coli and Helicobacter pylori.

TABLE 2
Similarity and identity of actinomycete GroEL2 amino acid
sequences to R. equi GroEL2

| | NCBI | | |
|---|---|---|---|
| | Accession | Similarity to | |
| | number of | R. equi GroEL | Identity to R. equi |
| Organism | GroEL2 protein | sequence | GroEL sequence |
| Tsukamurella | AAB499990 | 93.2% | 89.4% |
| tyrosinosolvens | | | |
| Streptomyces | 033658 | 89% | 83.3% |
| lividans | | | |
| Streptomyces | CAB93056 | 90.3% | 85.2% |
| coelicor | | | |
| Streptomyces albus | Q00798 | 90.5% | 85.4% |
| Mycobacterium | PO9239 | 93.4% | 89.6% |
| leprae | | | |
| Mycobacterium | PO6806 | 93.5% | 90% |
| tuberculosis H37Rv | | | |

Discussion

R. equi-related organisms such as Mycobacterium and Streptomyces sp. contain two groEL genes (Rinke de Wit, et al., 1992). Of these groEL1 is considered to be part of the groE operon whereas groEL2 is usually found at a different location on the chromosome (Duchêne. et al., 1994). The R. equi gene sequenced was identified as a groEL2 gene for the following reasons. Firstly, it was found to be homologous (90% identity) to other actinomycete groEL2 genes. Further, it did not appear to have a groES-like gene upstream from it suggesting it was not part of a groE operon. Previous studies on other R. equi related bacterial species such as Mycobacteria and Streptomyces have shown a similar arrangement of groEL genes (Duchêne, et al., 1994, Rinke de Wit, et al., 1992). It is likely that R. equi contains at least two GroEL encoding genes one of which is the monocistronic groEL2 sequenced in this study.

The high degree of identity that exists between the putative R. equi GroEL2 protein and the sequences of other actinomycete GroEL2 proteins is hardly surprising, as heat shock proteins are known to be highly conserved. Due to the highly conserved nature of these proteins and the genes encoding them, they are often used in bacterial phylogenetic studies (Gupta, 2000).

### Example 2

Development of Vaccine Candidates Against R. equi

Construction of groEL2 Based DNA Vaccine

The groEL2 gene was PCR amplified and cloned into vector pcDNA3 (Invitrogen) (Boshart, et al., 1985). The forward oligonucleotide primer containing a start codon was 5′-ACGGTACCATGGCCAAGATCATCGC-3′ [SEQ ID No 7] (KpnI site underlined; start codon in bold), the reverse oligonucleotide primer 5′-CTTCTAGACGGCGGATGCGAAATGC-3′ [SEQ ID No 8] (XbaI site underlined). The forward primer also contained a Kozak sequence, CCATGG (start codon underlined) (Kozak, 1982).

PCR was performed using standard conditions at an annealing temperature of 65° C., using a CsCl gradient purified preparation of pIMVS-Re1 as template and DyNAzyme™ EXT DNA polymerase (Finnzymes, Finland). The PCR product and pcDNA3 vector were digested separately with KpnI/XbaI and ligated together. The construct designated pcDNA3-Re1 (FIG. 3) was cloned into E. coli DH5 cc α for vaccine preparation.

Another groEL2 based DNA vaccine candidate, one that did not contain an ideal Kozak sequence was also constructed. This construct was created by digesting pIMVS-Re1 with KpnI/XbaI in order to obtain a fragment (approximately 2 kb, from 2714 bp to 4710 bp) containing the groEL2 gene. The fragment was ligated into KpnI/XbaI digested pcDNA3 vector. This construct was designated pcDNA3-hsp1 and was then cloned into E. coli DH5α for vaccine preparation.

Construction of vapA-Based DNA Vaccine

The vapA gene was PCR amplified and cloned into vector pcDNA3 (Invitrogen) (Boshart, et al., 1985). The forward oligonucleotide primer was: 5′-GA\overline{GGATCC}ATGGAGACTCTTCACAAGACG-3′ [SEQ ID No 9] (BamHI site underlined; start codon in bold) the reverse oligonucleotide primer was 5′-GAT\overline{GAATTC}TAACAACCGAGGCTGAGCG-3′ [SEQ ID No 10] (EcoRI site underlined).

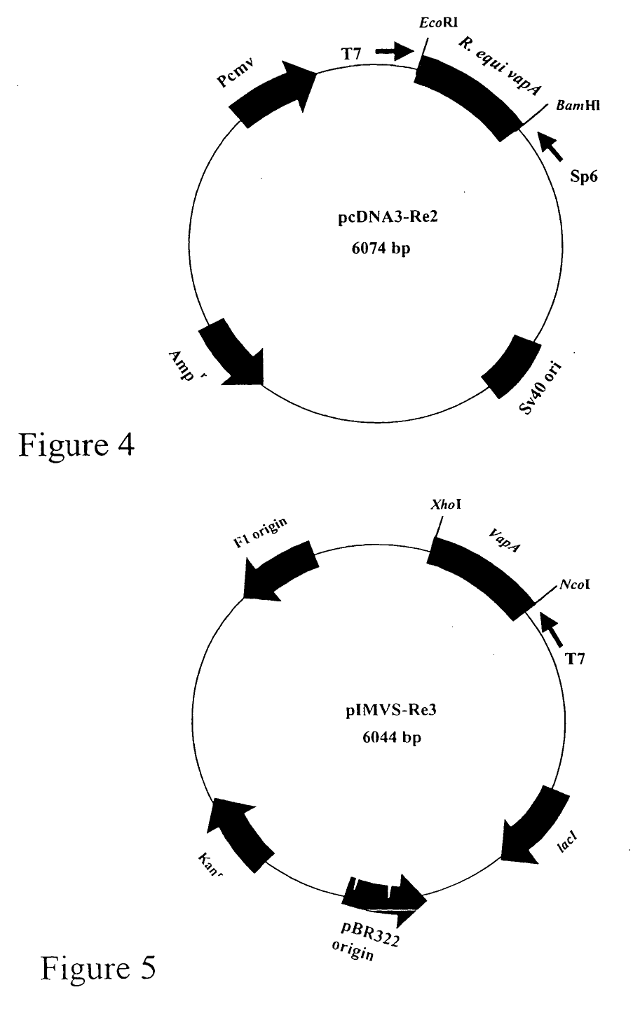

The forward primer also contained a Kozak sequence CC\overline{ATG}G (start codon underlined) (Kozak, 1982). PCR was performed using standard conditions at an annealing temperature of 65° C. using DyNAzyme™ EXT DNA polymerase (Finnzymes, Finland) and small scale plasmid extraction of R. equi ATCC 33701 as template. The PCR product and pcDNA3 vector were separately digested with BamHI/EcoRI and ligated together. The construct designated pcDNA3-Re2 was cloned into E. coli DH5 α for vaccine preparation (FIG. 4).

Another vapA based DNA vaccine candidate not containing a modified Kozak sequence was also constructed. This construct was created by inserting a PCR amplified vapA gene containing restriction sites EcoRI and BamHI into pcDNA3. The following oligonucleotides were used for PCR amplification of vapA: 5′-TCTTC\overline{GGATCC}GCTAATTACCGGC-3′ [SEQ ID No 11] (forward primer; BamHI site underlined) and 5′-G\overline{GAATTC}GCACCAATCCTGTTGCG-3′ [SEQ ID No 12] (reverse primer; EcoRI site underlined). Template used for the PCR reaction was a plasmid extraction of R. equi ATCC 33701 and PCR was performed using standard conditions at an annealing temperature of 55° C. using DyNAzyme™ EXT DNA polymerase (Finnzymes, Finland). Both the PCR product and pcDNA3 vector were digested separately with EcoRI and BamHI and ligated together. This construct was designated pIMVS-vap1 and cloned into E. coli DH5 cc for vaccine preparation.

Strategy to Enhance the Immunogenicity of VapA

In order to increase the immunogenicity of VapA as vaccine, the VapA B-cell epitope encoding genetic sequence was inserted into groEL2 to create a chimeric groEL21vapA construct. This approach has been used by other researchers who have shown that in chimeric gene constructs, the carrier gene acting as an adjuvant markedly enhances the immune response to the inserted epitope, thus circumventing the need for conventional adjuvants (Fomsgaard, et al., 1998).

The groEL2 gene was used as the carrier since previous studies have shown that heat shock proteins as carriers in conjugated vaccines can substantially enhance a T-cell mediated immune response to the conjugated antigen (Barrios, et al., 1992).

The eukaryotic expression vector pcDNA3 was employed in the construction of the DNA vaccines as it has been successfully used in other vaccine studies (Todoroki, et al., 2000, Turnes, et al., 1999). Importantly, his vector is known to be rich in immunostimulatory unmethylated cytosine-phosphate-guanine dinucleotide (CpG) sequences thought to promote the efficacy of plasmid vaccines (Cohen, et al., 1998) (Sato, et al., 1996, Strugnell, et al., 1997). Furthermore, plasmid DNA administered as an intramuscular vaccination is considered to activate CD4+ T-cells associated with a Th1 response (Leclerc, et al., 1997).

The protein vaccines used were tagged with histidine residues to enable convenient purification (using Ni-NTA agarose) of the protein in its native form following its expression in E. coli and is an approach that has been used by other researchers in the past for the preparation of protein vaccines (von Specht, et al., 2000).

Construction of Chimeric GROEL2/VAPA Based DNA Vaccine

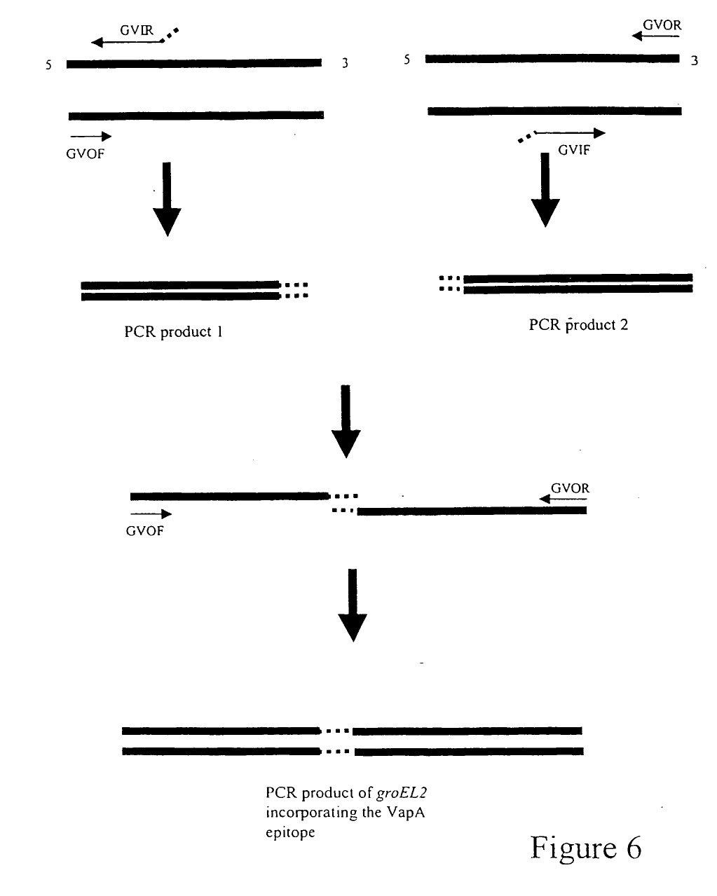

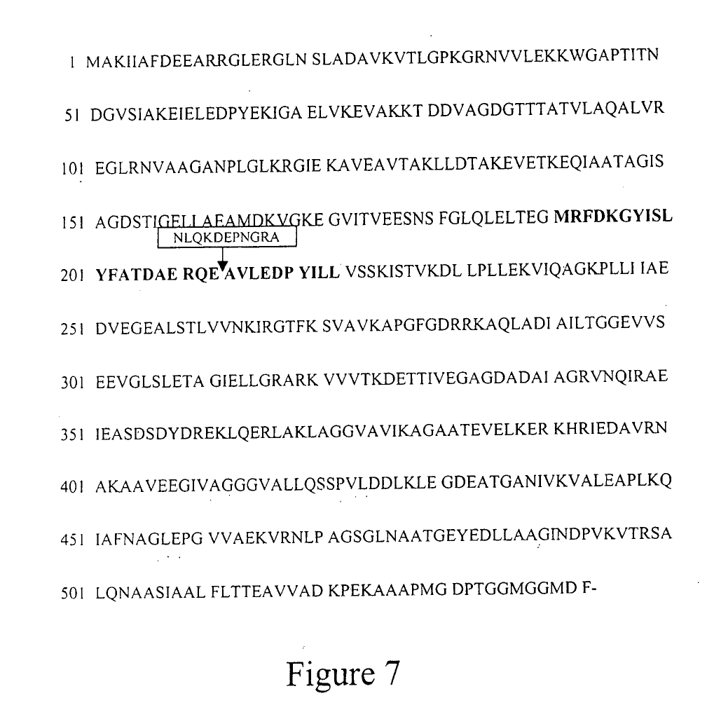

The chimeric groEL2/vapA vaccine construct was prepared by the insertion of the immunogenic epitope NLQKDEPNGRA [SEQ ID No 3] of VapA into a hydrophilic region (as indicated by the Hopp and Woods hydrophobicity plot) and a predicted immunogenic epitope of GroEL (based upon studies carried out on Salmonella typhi GroEL by Panchanathan) (Panchanathan, et al., 1998). The DNA sequence encoding the VapA epitope NLQKDEPNGRA [SEQ ID No 3], was inserted into groEL2 using overlap extension PCR mutagenesis (Ho, et al., 1989) (FIG. 6 and FIG. 7). Construct pcDNA3-Re1 was used as a template in the initial (two) PCR reactions.

Oligonucleotide primers used in one of these reactions were 5′-AACCTTCAGAAAGACGAACCGAACGGTCGAGCAGAGCGTCAGGAAGCGGTCC TCG-3′ [SEQ ID No 13] oligonucleotide primer GVIF (sequence corresponding to VapA epitope to be inserted is underlined) and 5′-CTATAGAATAGGGCCCTCTAGACGG-3′ [SEQ ID No 14]-oligonucleotide primer GVOR.

The other PCR was performed using oligonucleotide primers GVIR with sequence 5′-TGCTCGACCGTTCGGTTCGTCTTTCTGAAGGTTGGCGTCGGTCGCGAAGTACAGC G-3′ [SEQ ID No 15] (sequence corresponding to VapA epitope to be inserted is underlined) and oligonucleotide primer GVOF with sequence 5′-GAGACCCAAGCTTGGTACCATGG-3′ [SEQ ID No 16] (Kozak sequence underlined).

The PCR products obtained from both the above reactions were separated on a 1.5% agarose gel and purified using the QlAquick gel purification Kit (Qiagen, GmbH, Germany). Approximately 100 ng of each of the PCR products were used as the template in the final PCR reactions which were performed using oligonucleotide primers GVOF and GVOR (sequences described above).

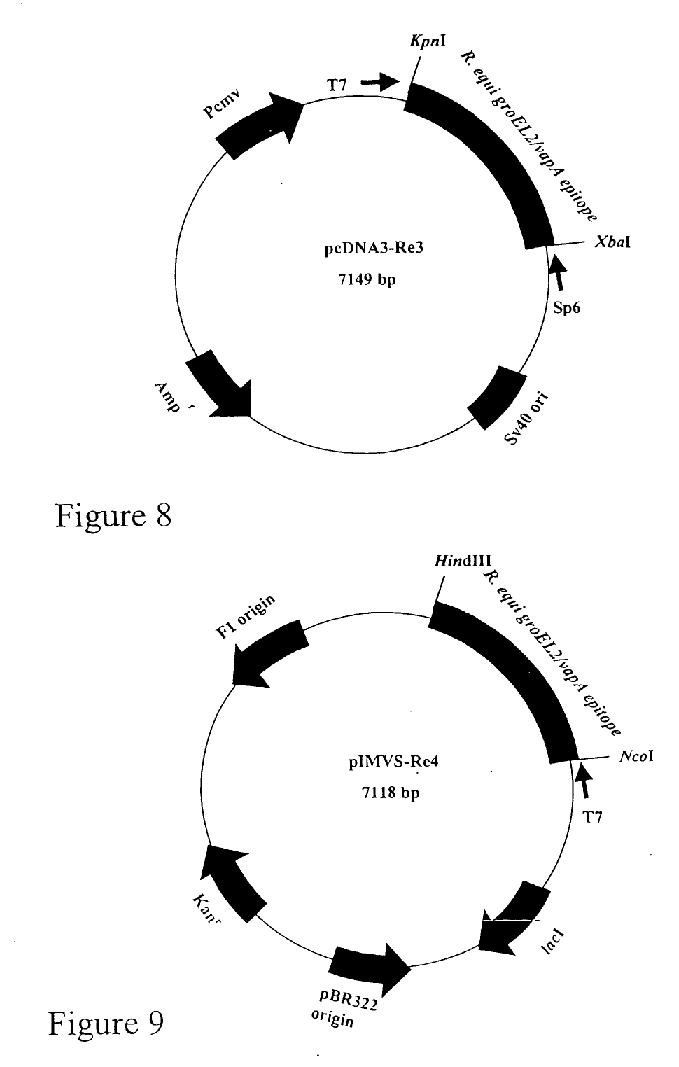

All PCR reactions were performed using standard conditions at an annealing temperature of 59° C. The PCR product and pcDNA3 vector were digested separately with KpnI/XbaI and ligated together. The construct was designated pcDNA3-Re3 and was cloned into E. coli DH5α for vaccine preparation (FIG. 8 and FIG. 9).

Preparation of DNA Vaccines

The vaccine constructs were propagated by growing a single colony containing the vaccine construct in a 10 ml aliquot of L-broth containing 100 μg/ml ampicillin for 6 hours at 37° C. with shaking. This culture was added to 500 nil L-broth containing 100 μg/ml ampicillin and grown overnight at 37° C. with shaking. The following day a large-scale plasmid extraction was performed. The plasmid extract was purified twice by CsCl gradient centrifugation and then dialysed overnight twice against 1×TE.

Prior to vaccine use, the plasmid preparation was processed using standard techniques (R. Strugnell, personal communication; protocol on DNA vaccine preparation, http://dnavaccine.com) as follows: NaCl (final concentration of 0.1M) and 2 volumes of absolute ethanol were added to the plasmid solution and mixed. The preparation was then precipitated at −20° C. for 30 mins. The DNA was pelleted by centrifugation for 15 mins at 17, 000 g. The pellet was washed with 70% ethanol, air dried and resuspended in 1×PBS (volume of PBS used was half that of the original volume of DNA preparation treated).

The vaccine preparation was then treated as follows in order to remove the contaminating endotoxin (Manthorpe, et al., 1993): Triton X-114 (TX-114) was added to the vaccine preparation to a final concentration of 1% (v/v) and mixed. The mixture was left on ice for 5 mins, then heated at 40° C. for 10 mins, allowing phase separation. The mixture was then centrifuged at 3000 g at 30° C. for 10 mins. The upper aqueous phase containing the DNA was removed and fresh TX-I 14 added and the extraction process was repeated twice. Finally, DNA was precipitated with the addition of an equal volume of isopropanol and centrifugation at 17,000 g, the DNA pellet was washed with 70% ethanol, dried and resuspended in 1×PBS (endotoxin free, Media Production Unit of the IMVS). The concentration of DNA was determined and was adjusted to 100 μg/l by diluting in 1×PBS, thereafter, 100 μl aliquots of the preparation were stored at −20° C. for vaccine use.

Endotoxin levels in the final vaccine preparations were confirmed to be less than 10 pg/ml by the QCL-1000 Limulus Amoebocyte Lysate Kit (BioWhittaker, Walkersville, Md., USA) (Li, et al., 1999) (test was performed by the Media Production Unit of the IMVS) prior to immunising the mice.

Expression of GROEL2-based DNA vaccine (pcDNA 3-Re1), VAPA-based DNA vaccine (pcDNA3-Re2) and chimeric GROEL2/VAPA DNA vaccine (pcDNA3-Re3) in Cos-7 cells Cos-7 cells were maintained in RPMI-1640 cell culture medium containing L-glutamine (CSL, KS, USA) and 10% foetal bovine serum (Sigma Chemical Co.). Twenty-four hours prior to transfection, cells were subcultured to ensure that they were in log growth phase. Approximately 3×10⁵ cells added to a Nunc™ 35 mm cell culture dish were transiently transfected, with pcDNA3, pcDNA3-hsp1, pcDNA3-vap1, pcDNA3-Re1, pcDNA3-Re2 or pcDNA3-Re3 vectors using the following method: A 15 μl aliquot of Fugene™ transfection reagent (Boehringer Mannheim) was diluted in 85 μl serum free cell culture media and incubated for 5 mins at room temperature. The preparations of each of the vaccine constructs and pcDNA3 vector (5 μg of CsCl gradient purified plasmid preparation) were added separately to the Fugene™ mixture and incubated for 15 mins at room temperature. The mixture was then added to the cells in fresh media and incubated at 37° C. in an incubator, in the presence of 5% CO₂ for 48 hours. Prior to harvesting, cells were checked for confluence. The growth medium was transferred to a centrifuge tube, 1 ml of 1×PBS added to the cells prior to collecting them from the bottom of the cell culture dish. The cells were added to the same centrifuge tube. The tube was then centrifuged at 10,000 g to pellet the cells. The pellet was washed by the addition of 1×PBS and centrifugation at 10,000 g. The pellet was finally resuspended in a 50 μl aliquot of 1×PBS, mixed with an equal volume of sample buffer and used in SDS-PAGE analysis and western immunoblot.

Preparation of a Plasmid Encoding His-Tagged VopA Protein

The vapA gene was amplified using PCR with a forward primer 5′-GAGGATCCATGGAGACTCTTCACAAGACG-3′ [SEQ ID No 17] containing anl introduced NcoI site (underlined) and reverse primer 5′-GCCTCGAGGGCGTTGTGCCAGCTACC-3′ [SEQ ID No 18] containing an introduced XhoI site (underlined) for cloning into pET-28a (+) (Novagen) expression vector. PCR was performed using standard conditions at an annealing temperature of 65° C. with plasmid extraction of R. equi ATCC 33701 as template and DyNAzyme™ EXT DNA polymerase (Finnzymes, Finland). The PCR product and vector were separately digested with NcoI and XhoI and ligated to create construct pIMVS-Re3 (FIG. 5).

His-tagged VapA Protein Preparation

His-tagged VapA from pIMVS-Re3 was essentially prepared using the method described for the production of His-tagged GroEL2, with the following modification: The protein was eluted from the Ni-NTA agarose using 100 mM EDTA (EDTA was used as imidazole could not be used to successfully elute the protein bound to Ni-NTA agarose). The protein eluate was dialysed twice against 1×PBS but was not subjected to endotoxin removal using TX-I 14 as this treatment was found to be ineffective for the removal of endotoxin from the VapA protein preparation, possibly due to the lipophilic nature of the protein. Endotoxin levels in the protein preparation were determined using the QCL-1000 Limulus Amoebocyte Lysate Kit (BioWhittaker, Walkersville, Md., USA) prior to use (testing carried out by the Media production Unit of the IMVS) and varied between 100-500 pg/ml. The His-tagged protein was separated on a 15% SDS PAGE gel and detected in a western immunoblot using the VapA specific monoclonal antibody (Takai, et al., 1993a).

Construction of a plasmid expressing His-tagged GroEL2/VapA protein The method of preparation of His-tagged GroEL2/VapA protein expressing chimeric gene was essentially that used for the production of groEL2/vapA in pcDNA3-Re3, with the following modification: instead of GVOR the following primer was used 5′-CGTCAAGCTTGAAGTCCATGCCGC-3′ [SEQ ID No 21] (HindIII site underlined), thereafter the His-tagged GroEL2VapA construct (pIMVS-Re4) was produced as described for pIMVS-Re2.

His-Tagged Chimeric GroEL21VapA Protein Preparation

The method of preparation of His-tagged GroEL2/VapA protein was essentially as described for His-tagged GroEL2 production. The purified protein was separated on a SDS PAGE gel and detected in a western immunoblot using GroEL2 specific monoclonal antibody.

Results

Expression of groEL2-Based DNA Vaccine in Cos-7 Cells

A large protein band of approximately 60 kDa was observed only in cells transfected with pcDNA3-Re1 indicating the production of GroEL2. This protein band was detected in western immunoblot analysis using the Chlamydia trachomatis Hsp60 specific monoclonal antibody.

Expression of VAPA based DNA vaccine in Cos-7 cells

A protein band of approximately 19 kDa and a larger diffuse band of approximately 15 kDa were expressed in cells transfected with pcDNA3-Re2 and was detected in western immunoblot analysis using VapA specific monoclonal antibody (Takai, et al., 1993a). Protein expression was not observed in Cos-7 cells transfected with pcDNA3-hsp2 (construct without an ideal Kozak sequence) or cells transfected with pcDNA3 vector. The expression of the 15 and 19 kDa proteins by pcDNA3-Re2 could not be observed in Coomassie Brilliant Blue stained SDS PAGE but only in a western immunoblot. This was not surprising as other workers have reported similar difficulty in visualising VapA on SDS PAGE using Coomassie Brilliant Blue stain (S. Takai, personal communication).

Expression of GROEL2/VAPA-Based DNA Vaccine in Cos-7 Cells

A large protein band of approximately 60 kDa was expressed in cells transfected with pcDNA3-Re3 (chimeric groEL2 IvapA vaccine construct). The protein was slightly larger than the GroEL2 protein expressed in Cos-7 cells.

### Example 3

### The Immunogenicity of R. equi Specific Vaccines as Determined in the Murine Model of Infection

Prior to vaccine use the protein preparations were processed as follows to make them suitable for in vivo use. All preparations were dialysed twice against 1×PBS. Endotoxin was removed from the GroEL2 and chimeric GroEL2/VapA protein preparations using TX-114. Endotoxin was not removed from the His-tagged VapA preparation as it was not possible to successfully remove the endotoxin from this preparation using the TX-114 based method.

Endotoxin levels were determined to be 100 pg/ml or less in the endotoxin treated protein preparations and around 100-500 pg/ml in the untreated His-tagged VapA protein preparations using the QCL-1000 Limulus Amoebocyte Lysate Kit (BioWhittaker, MD, USA).

Protein concentrations of the samples were determined using the Biorad protein assay and samples were stored in 100 μl aliquots at −20° C. until required. Prior to vaccination the sample was thawed at room temperature and diluted to a concentration of 2 mg/ml in 1×PBS.

Preparation of R. equi for use in a Live Vaccine and Challenge Studies

R. equi strain ATCC 33701 was prepared for infection of mice using previously described methods (Takai, et al., 1995a, Takai, et al., 1991 a). Prior to use in the animal studies, the R. equi strain was confirmed for the presence of the vapA gene by PCR and expression of VapA by western immunoblot. The strain was grown from an aliquot stored at −70° C. for 48 h in BHI broth, at 37° C. with shaking. Bacteria were pelleted by centrifugation at 10,000 g for 10 min washed once in 1×PBS and diluted in sterile saline to obtain a suspension giving an OD of approximately 0.6 at 550 nm. This suspension was diluted by 50% in sterile saline to obtain the final inoculum containing approximately 1.5×10⁷ organisms in a 100 μl aliquot. The suspension was further diluted in sterile saline to obtain a concentration of approximately 10⁵ organisms for use as a live vaccine. The approximate numbers of bacteria were confirmed in retrospect by plating an aliquot of the inoculum onto HBA just prior to inoculating the mice and counting the colonies following 48 h incubation at 37° C.

Mice used in the Study

Groups of 6-8 week old female BALB/c mice were used (five in each group). Animals were obtained from the Veterinary Services Division of the IMVS, (Gilles Plains, Adelaide, South Australia) and were certified to be specified pathogen free (SPF). Each group of mice was placed in separate filter top cages following immunisation.

DNA vaccination of Mice

Each group of mice was vaccinated with pcDNA3-Re1, pcDNA3-Re2, pcDNA3-Re3 or the pcDNA3 vector (control group). An aliquot of 50 μg of DNA (50 μl volume) was injected into each quadriceps muscle.

The animals were lightly anaesthetised via inhalation of Fluothane™ (Halothane) (Zeneca, Cheshire, UK) before being vaccinated. This was done for easy injecting of the animals as well as to prevent the injected DNA from being expelled by leg movement (muscle contraction). Animals were vaccinated on 3 occasions, 2 weeks apart.

Protein Vaccination of Mice

A 50 μl aliquot of the protein preparation containing a concentration of 100 μg protein, was mixed with an equal volume of 1.3% aluminium hydroxide gel (Alhydrogel, Asia Pacific Specialty Chemicals Ltd, NSW, Australia) to make up the 100 μl aliquot administered to each animal. Each group of mice was vaccinated with His-tagged GroEL2, chimeric GroEL2/VapA or VapA protein preparations. A control group of mice were vaccinated with 100 μl of 1×PBS. Animals were vaccinated intraperitoneally on 3 occasions, 2 weeks apart and bled prior to every boost and just before challenge.

Live R. equi Vaccination

Groups of mice were immunised with sub-lethal doses of live R. equi for comparison with the other vaccines. Animals were vaccinated with approximately 10⁵ live R. equi strain ATCC 33701 administered by the intraperitoneal route. Animals were vaccinated on three occasions, two weeks apart and bled prior to every boost and challenge. The number of organisms used in the vaccine was chosen based upon previous studies (Takai, et al., 1999a). Prior to vaccination an aliquot of the preparation was plated on HBA for retrospective determination of viable bacterial numbers in the preparation.

Co-administration of murine IL-12 encoding plasmid with DNA vaccines candidates The murine cytokine IL-12 expressing plasmid pORF-mIL12 (InvivoGen, CA, USA) (the plasmid is reported to secrete murine IL-12 by the manufacturer and was used without any modification) was electroporated into E. coli DH5a and DNA vaccine was prepared as previously described (see section on DNA vaccine preparation above) for intramuscular injection. The IL-12 insert contained the two murine IL-12 subunits (p35 and p40) encoding genes linked by 2 bovine elastin motifs (10 amino acids long), creating a single IL-12 open reading frame, ensuring the same level of expression of both subunits (Lee, et al., 1998). An aliquot of 5 μg of this preparation was co-injected (intramuscular) along with the antigen as previously described.

Obtaining Serum Samples from Mice

Two weeks after every immunisation and just before challenge blood samples were obtained from the mice by retro-orbital eye bleed. Prior to being bled, the animals were lightly anaesthetised via inhalation of Fluothane™ (Halothane) (Zeneca, Cheshire, UK). Blood samples from each group of mice were pooled (tubes containing the blood were incubated for 30 min at room temperature and then for 1 h at −4° C., finally they were centrifuged at 1000 g and the sera removed and stored at −20° C. until required.

ELISA for the detection of total IgG and immunoglobulin subclasses IGG1, IGG2a, IGG2b The pattern of IgG subclass produced during an immune response is widely accepted to be a reliable indicator of the type of cytokines produced in that response. Generally, IgG2a is considered to reflect the IFN-γ response (associated with a cell-mediated response) while IgG1 isotype switching is promoted by IL-4 (cytokine associated with humoral immunity) (Mosmann and Coffman, 1989).

The determination of the levels of IgG and IgG subclasses were performed as follows: Nunc™ maxisorp plates were coated with 5 μg/ml (100 μl aliquot per well) His-tagged GroEL2 or VapA in coating buffer (Na₂CO₃ 15 mM, NaHCO₃ 35 mM; pH 9.6) and used in an ELISA assay. Mouse serum was diluted 1 in 250 in PBS/0.05% Tween20 buffer containing 0.25 mg/ml E. coli extract (Promega, Wis., USA) and allowed to stand at room temperature for 30 mins prior to dispensing into the wells. The E. coli extract was used to aid in reduction of the background caused by the potential cross-reaction of any E. coli-specific antibodies present in the serum sample with the ELISA antigen. Secondary antibodies used were rabbit anti mouse IgG (H and L chain specific), γ2a, γ2b or yl chain specific peroxidase conjugated affinity purified monoclonal antibodies (Rockland, Pa., USA) at working dilutions of 1 in 5000, 1 in 4000, 1 in 5000 and 1 in 1000 respectively. The ODs of the reactions were read in an ELISA plate reader at a wavelength of 450 nm (reference wavelength 630 nm).

The results were confirmed by western immunoblot using His-tagged VapA and GroEL2 proteins (results not shown).

Preparation of Antigen for DTH Response Studies

R. equi strain ATCC 33701 was grown for 48 h in 500 ml BHI broth at 37° C. with shaking. The culture was pelleted by centrifugation at 10,000 g for 10 mins and washed twice in 1× PBS. The pellet was resuspended in 200-500 μl 1×PBS. The suspension was sonicated on ice for 30 secs and boiled for 10 mins. The protein concentration was determined and adjusted to 100 μg/ml by diluting in 1×PBS. This preparation was stored at −20° C. until required.

DTH Response Studies

Two separate studies (on groups of three mice immunised with the different vaccine candidates as described above) were carried out to measure DTH response in the hind footpads. The right hind footpad of each mouse was injected with 20 μl of the antigen and the corresponding left hind footpad was injected with 20 μl 1× PBS. Footpad thickness was measured at 24, 48 and 72 h intervals using Vernier calipers (the average of three readings ere obtained), DTH at 24 h was used in all analyses as the reaction was most significant at his time compared with the control. The percentage swelling was calculated using the following formula:
R. equi antigen footpad swelling (mm)-PBS footpad swelling/PBS footpad swelling×100
Statistical Analysis of Data

Data were analysed using a Wilcoxon (rank sum) two sample test at a significance level of P≦0.05. A non-parametric test was used as data were found not to be normally distributed. The data were analysed using SAS version 8.01 (SAS Institute, Inc. NC, USA).

Results

Symptoms Observed in Mice Following Challenge with R. equi

All animals except those immunised with the live R. equi vaccine developed symptoms of mild illness 24 hours after challenge. No significant weight loss was observed in these mice and none of the animals succumbed to the infection. The mice appeared to be completely normal by the fourth or fifth day after challenge.

Immune Response to Vaccination with Live R. equi

Mice vaccinated with live R. equi showed a Th1 biased immune response as indicated by moderately high IgG2a levels and low IgG1 levels. The IgG1, IgG2a and IgG2b responses increased with every boost. Interestingly, the VapA specific antibody response was higher than the GroEL2 specific response (Table 6.1). Importantly, a significant DTH response was also detected in these mice. Furthermore, the vaccinated mice showed enhanced clearance of R. equi following intravenous challenge.

Immune Response to GroEL2 Based Vaccine Candidates

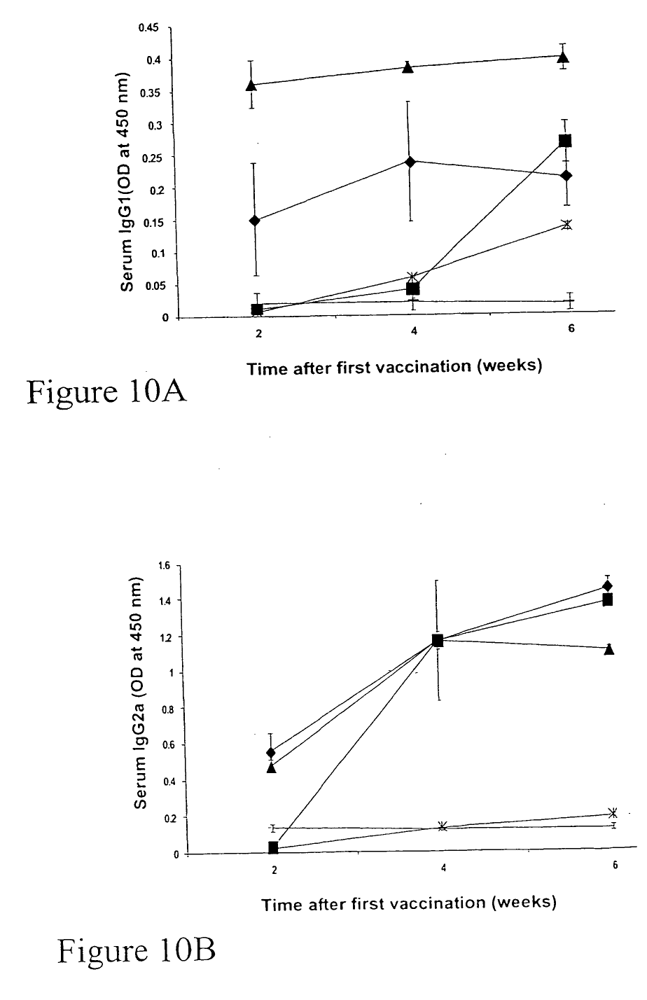

Significant levels of IgG2a antibodies to the R. equi GroEL2 protein were detected in mice vaccinated with both the His-tagged GroEL2 protein and DNA vaccine (pcDNA3-Re1), however, following two boosts the DNA vaccine was found to elicit a higher IgG2a response than the protein vaccine (FIG. 10B). Both the IgG1 (FIG. 10A) and IgG2a antibody (FIG.

FIG. 10B) responses progressively increased with every boost. Following the last boost the IgG2b response (FIG. 1 IA) was lower than the IgG2a response and higher than the IgG1 response for the DNA based vaccine.

The addition of pORF-mIL12 increased the IgG1 response after the last boost. In addition, following the last boost the IgG2b response was also increased. The IgG2a response was less following the last boost. The DTH response in the pORF-mIL12 co-immunised group was lower than that obtained with pcDNA3-Re1, indicating a lower Th1 bias than obtained with pcDNA3-Re1 alone.

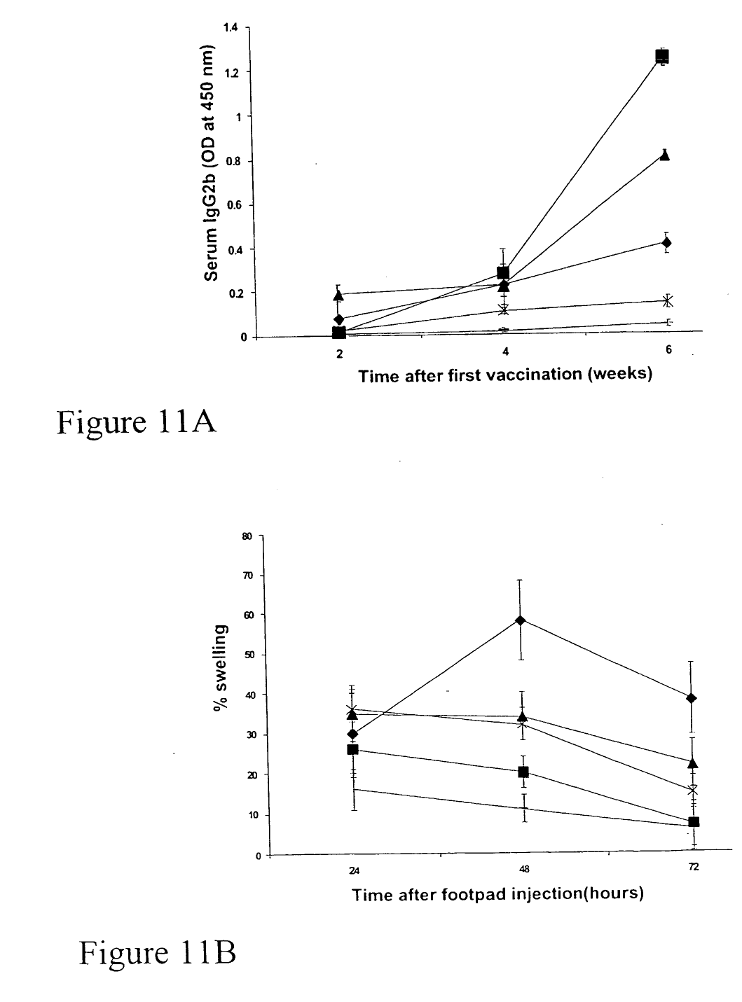

The DTH responses induced by the DNA and His-tagged protein vaccines were significant compared to the response in the mice immunised with the vector pcDNA3 (FIG. 11B). The response induced by the DNA vaccine pcDNA3-Re1 was higher than the response in the mice vaccinated with His-tagged GroEL2.

Immune Response to VapA Based Vaccine Candidates

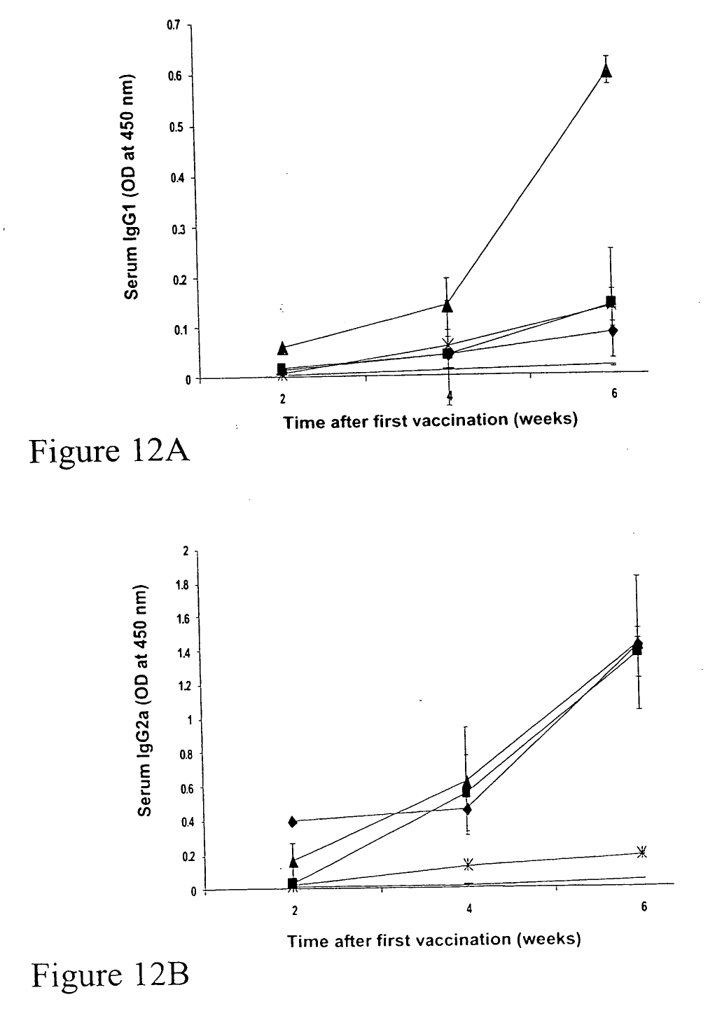

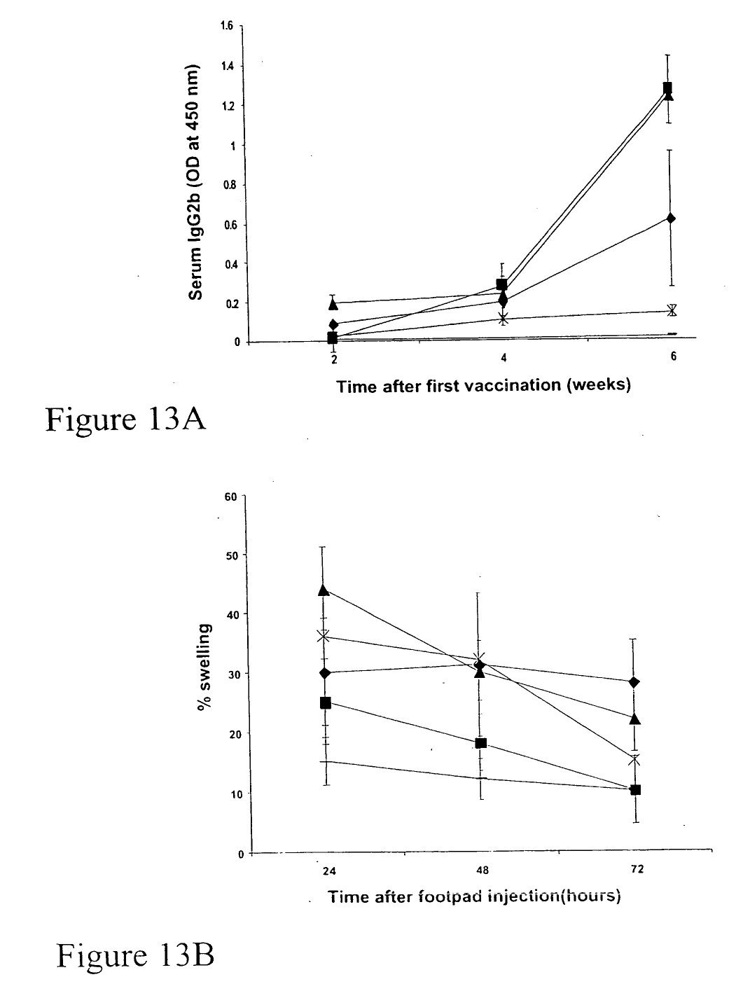

Moderate levels of IgG2a antibodies to the His-tagged VapA protein were detected in mice vaccinated with vapA based DNA vaccine (pcDNA3-Re2). The level of IgG1 antibodies was much lower than the IgG2a levels (FIG. 12A and FIG. 12B), indicating a Th1 biased immune response. The immune response to the His-tagged VapA vaccine was a higher IgG2a and a much higher IgG1 response than with the DNA vaccine, possibly indicating a weaker Th1 type bias in immune response than that observed with the DNA vaccine. The IgG2b response (FIG. 13A) was lower than the IgG2a and was similar or higher than the IgG1 response with both the DNA and protein vaccines tested, once again indicating a Th1 bias of the immune response. These results indicate a Th1 bias in the immune response elicited by the VapA based vaccines. The co-administration of pORF-mlLI2 substantially increased the IgG1 responses to pcDNA3-Re2 and also increased the IgG2a response but only until the last boost. The co-administration of pORF-mIL 12 did not significantly alter the IgG2b response.

The DTH response of the mice vaccinated with vapA DNA vaccine (pcDNA3-Re2) and His-tagged VapA vaccines was significantly higher than in the control mice (vaccinated with pcDNA3 alone) (FIG. 13B).

Immune Response to Chimeric GroEL2/VapA Vaccine Candidates

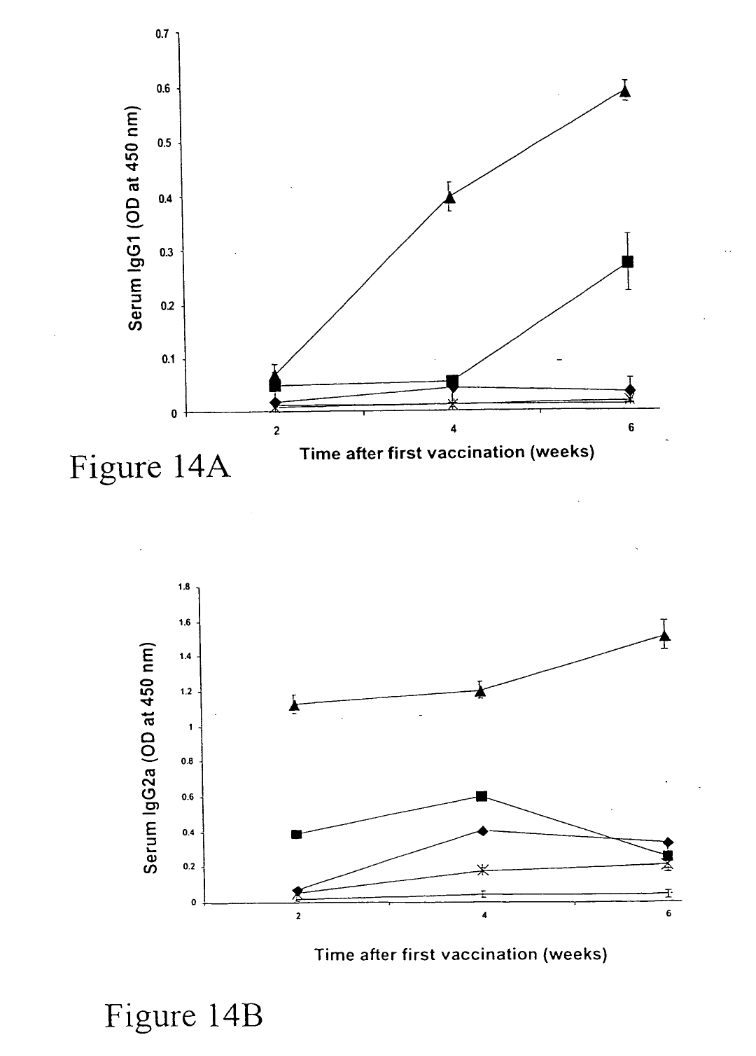

Significant levels of IgG2a antibodies to the R. equi GroEL2 protein were detected in mice vaccinated with the pcDNA3-Re3 (groEL2/vapA chimeric DNA vaccine) and chimeric GroEL/2NapA protein vaccines. Both the IgG1 and IgG2a antibody response progressively increased with every boost. The level of IgG1 antibodies was much lower than the IgG2a levels (FIG. 14A, FIG. 14B), indicating a Th1 bias in the immune response. The IgG2b response (FIG. 14A) was lower than the IgG2a and higher than the IgG1 response both for the DNA based and protein vaccines tested, suggesting a Th1 bias to the immune response.

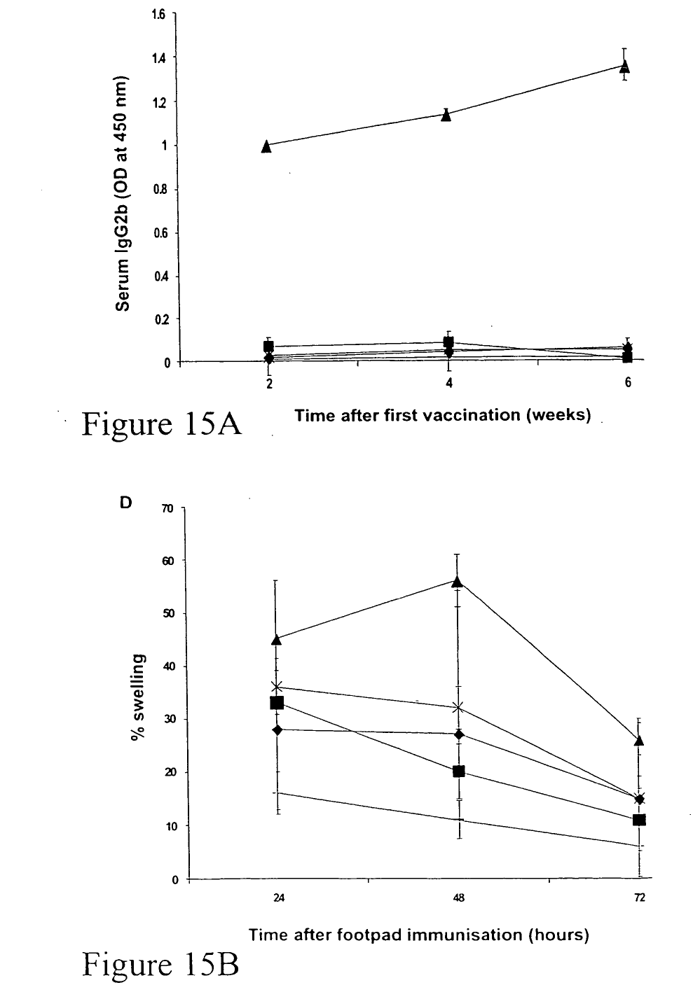

The DTH response of the mice vaccinated with pcDNA3-Re3 and His-tagged GroEL2/VapA was significantly greater than the control group mice vaccinated with pcDNA3 vector (FIG. 15B).

The addition of pORF-mIL 2 increased the IgG1 response but the IgG2a response was not significantly altered following the last boost. The IgG2b response was increased significantly following the final boost.

Assay to Detect Antibodies to the VapA B-Cell Epitope in the Chimeric groEL2/vapA Vaccine Construct.

Sera from mice immunised with the chimeric groEL2/vapA DNA vaccine (pcDNA3-Re3) were assayed to detect antibodies to the VapA B-cell epitope NLQKDEPNGRA [SEQ ID No 3]. This was carried using an ELISA with the biotinylated peptide NLQKDEPNGRA [SEQ ID No 3] as the target antigen. In addition, the His-tagged VapA was used as the target antigen in a separate ELISA. The OD values obtained with the sera from mice immunised with the chimeric groEL2/vapA based vaccines were not significantly different from that obtained with the sera from the control mice (results not shown). This suggests that the VapA epitope inserted into GroEL2 did not elicit a detectable IgG response in the mice.

Comparison of DNA Vaccines

Both the groEL2 (pcDNA3-Re 1) and chimeric groEL2/vapA (pcDNA3-Re3) vaccines produced significant total IgG, and specifically high IgG2a and DTH responses. The IgG1 and IgG2b responses were low to moderate. Generally, both the IgG1 and IgG2a responses were increased in the presence of pORF-mIL 2. The responses generated by pcDNA3-Rc1 and pcDNA3-Re3 were generally similar, except for the IgG1 response which was higher with pcDNA3-Re1. Not surprisingly, both the IgG1 and IgG2a antibody response progressively increased with every boost.

The immune response generated by the vapA based DNA vaccines was significantly lower than the other DNA vaccines, when pORF-mIL12 was co-injected there was a significant increase in IgG2a response following the first boost, however the response was still not as high as that observed with the groEL2/vapA (pcDNA3-Re3) chimeric vaccines. The IgG2a, IgG2b and IFN-γ response elicited by the groEL2 based vaccines (pcDNA3-Re1 and pcDNA3-Re3) were significantly higher than that observed with the live vaccine suggesting a significantly higher Th1 type immune response with the DNA vaccines. Contrary to this, the DTH response obtained with the live vaccine was significantly higher than that observed with any of the plasmid vaccines (Table 3). Importantly, none of the DNA vaccines elicited enhanced clearance in the mice unlike the live vaccine.

Comparison of Protein Vaccines

Generally, all three His-tagged protein vaccines elicited a predominantly IgG2a response however, the IgG1 response produced was also substantially higher than the response observed with the corresponding DNA vaccine candidates. This indicated a lower Th1 bias in the immune response elicited by the protein vaccines when compared with the DNA vaccines.

The His-tagged VapA vaccine elicited the strongest DTH response compared with the other protein vaccines tested. However, this result may be partly caused by the presence of relatively high levels of endotoxin (as a consequence of being expressed in a Gram negative bacterium) in the VapA preparation compared with the other vaccines. Interestingly, even the His-tagged GroEL2 and chimeric GroEL2 vaccines produced a significant DTH response that was higher than the response observed in the live R. equi immunised mice (Table 3).

The groEL2 based DNA vaccines (pcDNA3-Re1 and pcDNA3-Re3) appeared to elicit a strong Th1 type immune response as indicated by the IgG subclassing. In this regard, other reports have shown similar findings with groEL2 based DNA vaccines developed to other bacterial pathogens (Noll, et al., 1994). Importantly, these vaccines elicited an immune response that appeared to be more strongly Th1 biased than the vapA based vaccine (pcDNA3-Re2), suggesting that groEL2 was possibly a better DNA vaccine candidate than vapA.

The insertion of the VapA B-cell epitope NLQKDEPNGRA [SEQ ID No 3] into groEL2 appeared to have enhanced the Th1 response elicited by GroEL2, as observed in lower IgG1 and higher IgG2a responses.

The His-tagged protein vaccines unlike the corresponding DNA vaccines did not elicit a significant Th1 type immune response as indicated by a high IgG1 and IgG2a levels. Other researchers have reported similar findings with regard to the use of protein vaccines for intracellular pathogens, which require a Th1 response in the host for clearance of infection (Turner. et al., 2000).

The groEL2 based DNA vaccine was found to elicit an immune response that was more strongly Th1 biased than the vapA based DNA vaccine, an indication that groEL2 is more immunogenic than vapA, when administered as a DNA vaccine.

TABLE 3
Summary of immune response to vaccination with groEl2 based, vapA based and
live R. equi vaccines

| | DTH | ELISA OD (450 nm) (mean ± SD) | | |
|---|---|---|---|---|
| | response at | (antibody levels just prior to | | |
| Vaccine | 24 hours (% | challenge) | | |
| preparation | swelling ± SD)⁺ | Total IgG | IgG1 | IgG2a |
| groEL2 DNA | 30 ± 10 | 1.353 ± 0.10 | 0.337 ± 0.05 | 1.445 ± 0.05 |
| (pcDNA3-Re1) | | | | |
| His-tagged GroEL2 | 35 ± 7 | 1.581 ± 0.05 | 0.396 ± 0.08 | 1.100 ± 0.058 |
| protein | | | | |
| Chimeric | 30 ± 9 | 1.399 ± 0.12 | 0.084 ± 0.009 | 1.421 ± 0.19 |
| groEL2/vapA DNA | | | | |
| (pcDNA3-Re3) | | | | |
| His-tagged | 35 ± 7 | 1.468 ± 0.13 | 0.596 ± 0.02 | 1.413 ± 0.04 |
| GroEL2/VapA | | | | |
| protein | | | | |
| vapA DNA | 28 ± 15 | 0.716 ± 0.13 | 0.035 ± 0.02 | 0.33 ± 0.007 |
| (pcDNA3-Re2) | | | | |
| His-tagged VapA | 45 ± 11 | 1.479 ± 0.10 | 0.590 ± 0.02 | 1.153 ± 0.08 |
| protein | | | | |
| Live R. equi (10⁵ | 36 ± 5.2 | VapA | 0.019 ± 0.001* | 0.206 ± 0.02* |
| organisms) | | specific | | |
| | | 0.438 ± 0.019* | | |
| | | GroEL2 | | |
| | | specific | | |
| | | 0.148 ± 0.018** | 0.136 ± 0.002** | 0.186 ± 0.02** |
| Control (pcDNA3 | 16 ± 4 | 0.134 ± 0.019 | 0.013 ± 0.002 | 0.038 ± 0.021 |
| vector) | | | | |
| Control (1 × PBS)‡ | 14 ± 8 | 0.104 ± 0.01 | 0.015 ± 0.002 | 0.034 ± 0.001 |

⁺Preliminary studies indicated that DTH responses measured at 24 hours after footpad injection showed the greatest difference among the vaccine preparations
*VapA antigen specific response
**GroEL2 antigen specific response
‡As there was no significant difference between the overall response in PBS and pcDNA3 control mice, the PBS control data were not included in further statistical analyses

### REFERENCES

Altschul et al., (1.997) Nucleic Acids Res. 25:3389-3402.
Babiuk et al., (I 998). Curr Top Microbiol Immunol. 226:90-106.
Bachy et al., (2001). Vaccine. 19:1688-1693.
Barrios et al., (1992). Eur Immunol. 22:1365-1372.
Barton and Hughes. (1980) Vet Bull. 50:65-80.
Barton (1992) Equine Infectious Diseases VI ed. Plowright et al., R&W publications (Newmarket) Ltd. p77.
Barton and Hughes. 1984. Vet Microbiol. 9:65-76.
Becú et al., (1997). Vet Microbiol. 56:193-204.
Behr and Small. (1997). Nature. 389:133-134.
Boshart et al., (1985). Cell. 41:521-530.
Capdevila et al., (1997). Scand J Infect Dis. 29:535-541.
Chaplin et al., (1999). Infect Immun. 67:6434-6438.
Cohen et al., (1998). Modulating the immune response to genetic immunization. FASEB J. 12:1611-1626.
Craig et al., (1993). Microbiol Rev. 57:402-414.
Darquet et al (1999). Gene Ther. 6:209-218.
de León et al., (1997). Microbiology. 143:3563-3571.
Dietrich et al., (2001). Vaccine. 19:2506-2512.
Duchéne et al., (1994). Gene. 144:97-101.
Fomsgaard et al., (1998). Scand J Immunol. 47:289-295.
Giguère et al., (1999a). Infect Immun. 67:3548-3557.
Giguère and Prescott. (1997). Vet Microbiol. 56:313-334.
Goodfellow (1987). Vet Microbiol. 14:205-209.
Gupta (11995). Mol Microbiol. 15: 1-11.
Gupta (2000). FEMS Microbiol Rev. 24:367-402
Hondalus and Mosser. (1994). Infect Immun. 62:4167-4175.
Hopp and Woods. (1981). Proc. Natl. Acad. Sci. U.S.A. 78;3824-3828
Kamath et al., (1999). Immunology. 96:511-516.
Karlin and Brocchieri. (2000). Proc Natl Acad Sci U SA. 97:11348-11353.
Kim et al., (2001). Immunology. 102:234-241.
Kozak (1982). J. Mol. Biol. 156:807-820.
Lathigra et al., (1991). Curr Top Microbiol Immunol. 167:125-143.
Leclerc et al., (1997). Cell Immunol. 179:97-106.
Lee et al., (1998). Hum Gene Ther. 9:457-465.
Li et al., (1999) Infect Immun. 67:4780-4786.
Lowrie et al., (1997). Vaccine. 15:834-838.
Lowrie et al., (1.999) Nature 400; 269
Ma et al., (2000). A dynamic model for the allosteric mechanism of GroEL. J. Mol. Biol. 302:303-313.
Manthorpe et al., (1993). Hum Gene Ther. 4:419-431.
Mazodier et al., (1991). J. Bacteriol. 173:7382-7386.
Mosmann and Coffman. (1989). Annu Rev Immunol. 7:145-173.
Noll et al., (1994). Infect Immun. 62:2784-2791.
Pal et al., (1999). Vaccine. 17:459-465.
Panchanathan et al., (1998). Immunol. lett. 62:105-109.
Park and Scott. (2001). Scand J Immunol. 53:529-532.
Prescott et al., (1997a). Am J Vet Res. 58:356-359.
Prescott et al., (1997b). Vet Microbiol. 56:213-225.
Ramshaw and Ramsay. (2000). Immunol Today. 21:163-165.
Richardson et al., (1998). TIBS 23:138-143
Rinke de Wit et al., (1992). Mol Microbiol. 6:1995-2007.
Ruimy et al., (1995). Int J Syst Bacteriol. 45:740-746.
Saibil. (2000). Curr. Opinions in Structural Biology. 10:251-258
Sambrook, et al., (1989). Molecular Cloning: a laboratory manual, 2nd ed. Cold Spring
Harbor Laboratory Press, Cold Spring Harbor, N.Y., USA.
Sampson et al., (1986). J Clin Microbiol. 23:92-99.
Sato et al., (1996). Science. 273:352-354.
Segal, and Ron. (1996). FEMS Microbiol Lett. 138:1-10.
Shinnick (1991). Curr Top Microbiol Immunol. 167:145-160.
Smola et al., (1994) J Appl Bacteriol. 77:325-333.
Strugnell et al., (1997). Immunol Cell Biol. 75:364-369.
Svanholm et al., (2000). Scand J Immunol. 51:345-353.
Takai (1997). Vet Microbiol. 56:167-176.
Takai et al., (1993a). J Clin Microbiol. 31:2780-2782.
Takai et al., (1995a). J Clin Microbiol. 33:1624-1627.
Takai et al., (1999a). FEMS Immunol Med Microbiol. 24:1-9.
Takai et al., (1991 a). J Clin Microbiol. 29:439-443.
Toda et al., (1997). Immunology. 92:111-117.
Todoroki et al., (2000). Biochem Biophys Res Commun. 277:159-163.
Turner et al., (2000). Infect Immun. 68:3674-3679.
Turnes et al., (1999). Vaccine. 17:2089-2095.
Vanrompay et al., (1999). Clin Exp Immunol. 118:49-55.
Vanniasinkam et al (2001) J Clin Microbiol 39(4) 1633-1637
Varga et al., (1997). Vet Microbiol. 56:205-212.
von Specht et al., (2000). J Biotechnol. 83:3-12.
Yoshida et al., (2000). Vaccine. 18:1725-1729.
Zügel and Kaufmann. (1999b). Clin Microbiol Rev. 12:19-39.

1. A chimeric protein, said chimeric protein being a GroEL protein, modification or analogue thereof having a surface exposed exogenous amino acid sequence inserted therein, the exogenous amino acid sequence configured to elicit an immune response specifically reactive to the antigenic determinant.

2. The chimeric protein as in claim 1 wherein the exogenous amino acid sequence is inserted in a hydrophilic region of the GroEL protein.

3. The chimeric protein as in claim 1 wherein the exogenous amino acid sequence is inserted into a location of the GroEL protein comprising a GroEL antigenic determinant.

4. The chimeric protein as in claim 2 wherein the GroEL is derived from R. equii.

5. The chimeric protein as in claim 4 wherein the hydrophilic region is selected from the group of hydrophobic regions consisting of V26-S54, V73-T90, G109-A155, M191-L246, R270-I290, G342-A 197, and V 415-N468.

6. The chimeric protein as in claim 4 wherein the hydrophilic region is M191-L246.

7. The chimeric protein as in claim 1 wherein the exogenous amino acid sequence has a length of in the range of 3 to 25 amino acids.

8. The chimeric protein as in claim 1 wherein the exogenous amino acid sequence has a length of about 11 amino acids.

9. The chimeric protein as in claim 1 wherein the exogenous amino acid sequence includes an immunodominant antigenic determinant of a pathogenic bacterial species.

10. The chimeric protein as in claim 9 wherein the GroEL protein is derived from the pathogenic bacterial species.

11. The chimeric protein as in claim 9 wherein the pathogenic species is Rhodococcus equii and the immunodominant antigenic determinant is derived from the Vap A protein.

12. The chimeric protein as in claim 11 wherein the antigenic determinant is present in SEQ ID No 2.

13. The chimeric protein as in claim 11 wherein the antigenic determinant is present in SEQ ID No 3.

14. A nucleic acid molecule including a chimeric protein encoding sequence and a control element positioned for expression of said chimeric protein, said chimeric protein being a GroEL protein, modification or analogue thereof having a surface exposed exogenous amino acid sequence inserted therein, the exogenous amino acid sequence configured to elicit an immune response specifically reactive to the antigenic determinant.

15. The nucleic acid molecule as in claim 14 wherein the exogenous amino acid sequence is inserted in a hydrophilic region of the GroEL protein.

16. The nucleic acid molecule as in claim 14 wherein the exogenous amino acid sequence is inserted into a location of the GroEL protein comprising a GroEL antigenic determinant.

17. The nucleic acid molecule as in claim 15 wherein the GroEL protein is derived from R. equi.

18. The nucleic acid molecule as in claim 14 wherein the exogenous amino acid sequence has a length in the range of 3 to 25 amino acids.

19. The nucleic acid molecule as in claim 14 wherein exogenous amino acid sequence includes an immunodominant antigenic determinant of a pathogenic bacterial species.

20. The nucleic acid molecule as in claim 19 wherein the GroEL protein is derived from the pathogenic bacterial species.

21. The nucleic acid molecule as in claim 19 wherein the pathogenic species is Rhodococcus equii and the immunodominant antigenic determinant is derived from the Vap A protein.

22. The nucleic acid molecule as in claim 21 wherein the antigenic determinant is present in SEQ ID No 2.

23. The nucleic acid molecule as in claim 14 wherein the chimeric protein also includes a non GroEL sequence that assists in the purification of the protein.

24. The nucleic acid molecule as in claim 23 wherein a plurality of histidine residues are added to the C terminus of the chimeric protein.

25. The nucleic acid molecule as in claim 14 including a promoter for expression in a host cell to elicit the immune response.

26. The nucleic acid molecule as in claim 25 said DNA molecule vector encoding a co-stimulatory molecule, said co-stimulatory molecule capable of stimulating the immune response of the host.

27. A method of eliciting an immune response in a mammal against an antigenic determinant the method including the step of administering to the mammal a chimeric protein, the chimeric protein being a GroEL protein, modification or analogue thereof having a surface exposed exogenous amino acid sequence inserted therein, said exogenous amino acid sequence configured to elicit an immune response specifically reactive to the antigenic determinant.

28. The method of eliciting an immune response as in claim 27 wherein the exogenous amino acid sequence is inserted in a hydrophilic region of the GroEL protein.

29. The method of eliciting an immune response as in claim 27 wherein the exogenous amino acid sequence is inserted into a location of the GroEL protein comprising a GroEL antigenic determinant.

30. The method of eliciting an immune response as in claim 27 wherein the exogenous amino acid sequence has a length in the range of 3 to 25 amino acids.

31. The method of eliciting an immune response as in claim 27 wherein exogenous amino acid sequence includes an immunodominant antigenic determinant of a pathogenic bacterial species.

32. The method of eliciting an immune response as in claim 31 wherein the GroEL protein is derived from the pathogenic bacterial species.

33. The method of eliciting an immune response as in claim 31 wherein the pathogenic species is Rhodococcus equii and the immunodominant antigenic determinant is derived from the Vap A protein.

34. The method of eliciting an immune response as in claim 33 wherein the antigenic determinant is included in SEQ ID No 2.

35. The method of eliciting an immune response as in claim 28 wherein the GroEL protein is derived from R. equii.

36. The method of eliciting an immune response as in claim 27 wherein the immune response includes an antibody response specific to the antigenic determinant.

37. The method of eliciting an immune response as in claim 36 wherein the antibody response is proportionately greater for IgG2a in comparison to IgG1.

38. The method of eliciting an immune response as in claim 27 wherein the chimeric protein is administered in purified form in a pharmaceutically acceptable carrier.

39. The method of eliciting an immune response as in claim 38 wherein an adjuvant is co-administered.

40. The method of eliciting an immune response as in claim 27 wherein a nucleic acid molecule capable of expressing the chimeric protein is administered to the mammal so that on insertion into a cell of the mammal expression in the host the chimeric protein is expressed in the cell.

41. The method of eliciting an immune response as in claim 40 wherein the nucleic acid also expresses an immunostimulatory molecule in the cell.

42. The method of eliciting an immune response as in claim 40 wherein the nucleic acid is administered by intramuscular injection.
